(19) United States
(12) Patent Application Publication
(10) Pub. No.: US 2017/0267689 A1
(43) Pub. Date: Sep. 21, 2017

(54) STIMULI-SWITCHABLE MOIETIES, MONOMERS AND POLYMERS INCORPORATING STIMULI-SWITCHABLE MOIETIES, AND METHODS OF MAKING AND USING SAME

(71) Applicant: THE GOVERNORS OF THE UNIVERSITY OF ALBERTA, Edmonton (CA)

(72) Inventors: Carlo David MONTEMAGNO, Edmonton (CA); Sinoj ABRAHAM, Edmonton (CA); Surjith Kumar KUMARAN, Edmonton (CA)

(21) Appl. No.: 15/506,695

(22) PCT Filed: Aug. 25, 2015
(86) PCT No.: PCT/CA2015/050812

§ 371 Date: Feb. 24, 2017

Related U.S. Application Data
(60) Provisional application No. 62/041,593, filed on Aug. 25, 2014.

(51) Int. Cl. C07D 491/18; C08G 69/00; C09K 9/02; C08F 120/36; C08F 120/32; C08G 63/08; C08F 8/32

(52) U.S. Cl. CPC C07D 491/18; C08G 63/08; C08G 69/00; C08F 8/32; C08F 120/36; C08F 120/32; C09K 9/02; C09K 2211/1466; C09K 2211/145; C09K 2211/1088; C09K 2211/1029

(57) ABSTRACT

Stimuli-switchable moieties, monomers incorporating stimuli-switchable moieties, and polymers incorporating such stimuli-switchable moieties are provided. The stimuli-switchable moiety can be a pyrano aryl chromenone-derivative. The stimuli-switchable monomer can be a lactone monomer. The stimuli-switchable monomer can be an amino acid, which can be incorporated into a specific peptide sequence by peptide synthesis.

### REFERENCE TO RELATED APPLICATIONS

This application claims priority to and the benefit of U.S. provisional patent application No. 62/041,593 filed 25 Aug. 2014, the entirety of which is incorporated by reference herein for all purposes.

### TECHNICAL FIELD

Some embodiments of the present invention relate to moieties and/or molecules (e.g., monomers and polymers) capable of changing their physical and/or chemical properties in response to external stimuli. Some embodiments of the present invention pertain to polymers and/or surfaces incorporating such molecules.

### BACKGROUND

The construction of dynamic materials, i.e. materials that can change their structure and/or properties in response to environmental stimuli, is an area of recent interest. Dynamic materials have potential application in fields such as intra-cellular delivery, including gene delivery, rewritable data storage, optical switching, chemical sensing, holography, microfluidics, self-cleaning surfaces, smart windows, protective coatings, tissue engineering, and protein chromatography or controlled bioadhesion.

Unlike other physical stimuli such as temperature and pH, light stimuli can be used remotely with spatiotemporal control, and therefore need not effect change on a whole system. Also photo-switchable surfaces can be activated by a selected wavelength range and deactivated by an additional selected wavelength range. Given these unique properties, photo-responsive polymers have been used in nanotechnology and biotechnology.

One example of a class of molecules that can undergo a reversible change in structure and properties in response to external stimuli are spiropyrans, which have the general chemical structure (10) shown below:

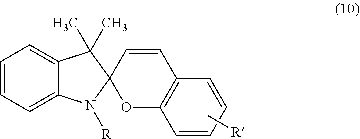

Spiropyrans can undergo structural isomerization in response to a variety of stimuli, including light, temperature, metal ions, redox potential, or mechanical stress (see e.g. Klajn, 2014). Spiropyrans can reversibly switch between a closed-ring (spiropyran) (11) or leuco form and an open-ring (merocyanine) (12) form when subjected to ultraviolet (UV) radiation (λ=365 nm) or near-infrared (NIR) radiation, as shown below, and can be reversibly switched back to the closed-ring form by the application of heat or visible light. The closed-ring form of spiropyran is hydrophobic, while the open-ring form is hydrophilic. Spirooxazines are closely related molecules that can also undergo structural isomerization between a spirooxazine and a merocyanine form upon the application of UV light, which can be reversed by the application of heat or visible light, as shown in Scheme 1.

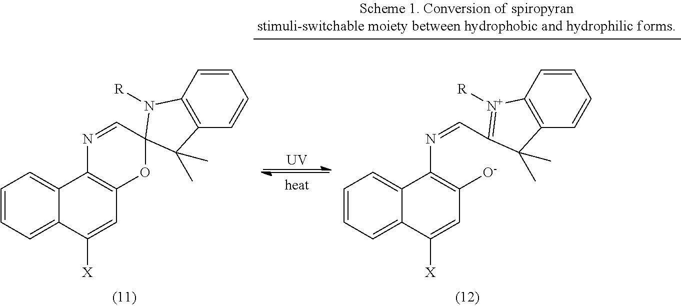

Another example of a class of molecules that can undergo a reversible change in structure and properties in response to external stimuli are azobenzenes, which have the general structure (13) shown below:

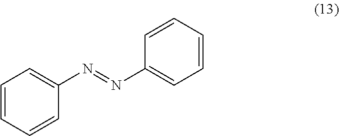 [SMILES: c1ccc(/N=N/c2ccccc2)cc1]

Azobenzene and its derivatives undergo photoisomerization of trans (14) and cis isomers (15). The two isomers can be switched with particular wavelengths of light: ultraviolet light for the conversion from trans to cis, and blue light for the conversion from cis to trans, as shown below in Scheme 2:

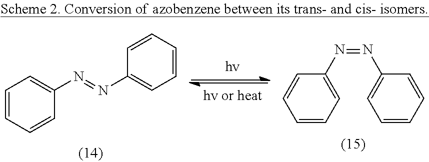 [SMILES: c1ccc(/N=N/c2ccccc2)cc1 ; c1ccc(/N=N\c2ccccc2)cc1]

An example of a class of molecules that can undergo an irreversible cleavage upon exposure to light are molecules incorporating coumarins having the general structure (16) shown below:

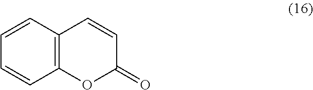 [SMILES: O=c1ccc2ccccc2o1]

Another example of a molecule that can undergo an irreversible change in structure upon exposure to light is molecules incorporating a nitrobenzene moiety having the general structure (17) shown below:

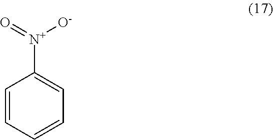

Other known photoswitchable moieties include bisthienylethenes having the general structure (18), flugides having the general structure (19), 2-diazo-1,2-naphthoquinone-5-sulfonyl-methylacrylamide having the structure (20), as shown below:

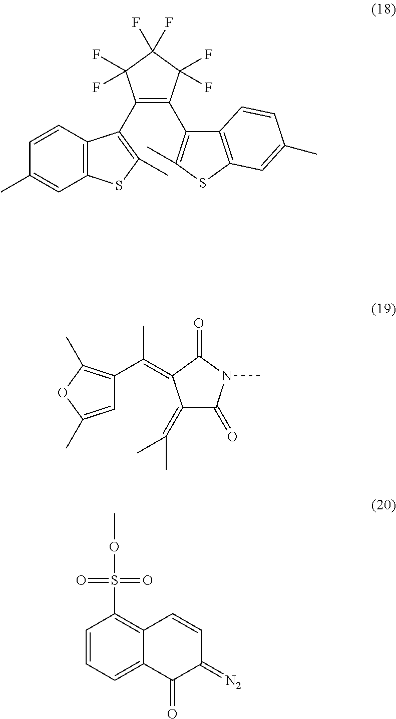

Methods of producing polymeric surfaces incorporating photochromic moieties have been developed. In previous approaches, a photo-switchable surface is prepared by grafting stimuli-responsive agents to immobilize them on a surface. There is a need for other methods of producing polymeric surfaces incorporating photochromic moieties, including in some applications a need for other methods of producing polymeric surfaces that can enable specific control over the location of a stimuli-switchable moiety within the polymer.

There is also a need for switchable moieties that can switch more strongly between a to hydrophobic state and a hydrophilic state, so that more sensitive or more useful surfaces bearing polymers incorporating such switchable moieties can be produced.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides a polymer having a plurality of monomeric units. The monomeric units can be napthacene derivatives or lactone derivatives. At least some of the monomeric units comprise a stimuli-switchable moiety. The polymer is switchable in response to an external stimuli.

In some aspects, the napthacene derivative can have the structure (I), (II) or (III):

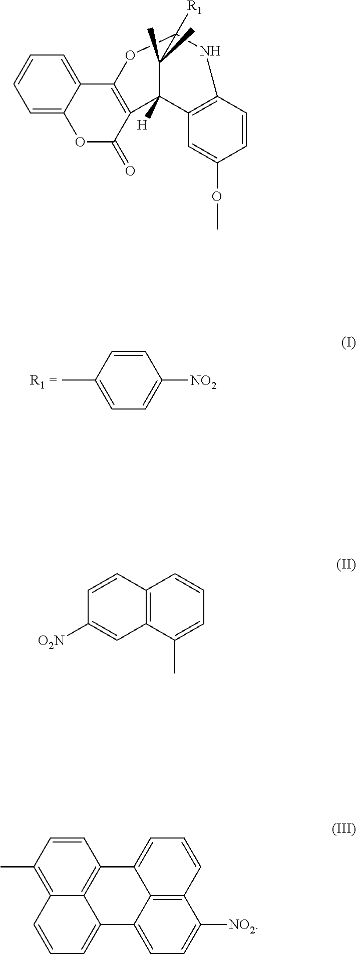

The napthacene derivative can reversibly switch from hydrophobic to hydrophilic upon exposure to light having a wavelength in the range of approximately 250 nm to 450 nm or approximately 650 nm to 800 nm. The napthacene derivative can reversibly switch from hydrophilic to hydrophobic, upon an increase in temperature to above approximately 50° C.

In some aspects, the monomeric units are lactone derivatives. In some aspects, a polymerizable monomer having a stimuli-switchable moiety wherein the monomer is a lactone is provided. The lactone can be butyrolactone, valerolactone, caprolactone, nonalactone, or any other suitable lactone. The stimuli-switchable moiety can be provided at any position on the lactone ring. The carbons of the lactone ring may have additional substituents. One aspect provides a polymer produced by the ring-opening polymerization of a plurality of lactone monomers bearing a stimuli-switchable moiety.

In one aspect, a method of synthesizing a peptide incorporating at least one stimuli-switchable moiety is provided. A plurality of amino acids are provided, at least one of the amino acids being a modified amino acid incorporating a stimuli-switchable moiety. A first one of the plurality of amino acids is coupled to a solid support. A second one of the plurality of amino acids is coupled to the first one of the plurality of amino acids. Each successive amino acid is coupled to the preceding amino acid to produce a peptide having a desired sequence. At least one of the amino acids that is a modified amino acid is incorporated into the peptide at a desired location during synthesis of the peptide.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 shows the color change from colourless (left image) to purple colored (right image) experienced by one exemplary stimuli-switchable moiety upon the application of light.

FIG. 2 shows the change in contact angle of a droplet on a surface incorporating a stimuli-switchable moiety when the stimuli-switchable moiety is changed from its hydrophobic (left panel) to its hydrophilic (right panel) configuration using light.

FIG. 3 shows the hypothetical generation of a predetermined pattern on a polymeric surface incorporating a photoswitchable moiety.

FIG. 4 shows different photoswitchable moieties that can be switched from a first configuration to a second configuration by the application of light having different wavelengths.

FIG. 5 shows the change in contact angle of a droplet on a surface incorporating a stimuli-switchable moiety when the stimuli-switchable moiety is changed from its hydrophobic (left panel) to its hydrophilic (right panel) configuration by the application of light.

FIG. 6 shows the results of atomic force microscopy used to visualize the interaction of cationic silica nanoparticles with a surface bearing a photoswitchable moiety.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

Some embodiments of the present invention relate to “schizophrenic” or “dynamic” dual demeanor polymeric surfaces with the capability of switching properties, such as from hydrophilic to hydrophobic, either reversibly or irreversibly, when induced by external stimuli like light, heat, pressure, electric stimulus or chemical stimulus (e.g. a change in pH or the presence of particular ions). Some embodiments of the present invention relate to methods for preparing such polymeric surfaces using functional monomers such as acrylic monomers, N-carboxy anhydrides of amino acids or modified amino acids, their polymerizations and surface grafting using controlled radical polymerizations (eg: Atom Transfer Radical Polymerization (ATRP), Reversible Addition Fragmentation Polymerization (RAFT), Nitroxide Mediated Polymerization (NMP)), Ring opening Polymerization (ROP), NCA (N-carboxyanhydride)) or solid phase peptide synthesis producing polymers and peptides. These polymeric surfaces exhibit reversible and/or irreversible transformations, for example, by changing surface hydrophobicity, color, ionic charges, pH etc. enabling them to be utilized for microfluidic devices, display devices and optical and thermal memory devices.

Polymers possessing the ability to alter their properties with a change in environmental stimuli can be utilized for wide variety of applications such as drug delivery agents, biocatalysts, membranes, selective size separations, transducers, display devices, microfluidics, optical memory devices, thermal memory devices, and the like. Surfaces can be generated which can exhibit reversible or irreversible transformations by changing their surface hydrophobicity, colour, ionic charge, pH, and the like. Polymers with photo-sensitive moieties can offer an unprecedented combination of physical properties including photo-switching, polarity switching, color changing, phase transition of liquid, and the like, mainly due to a trans- to cis isomerization occurring upon irradiation. Some of the moieties can ring close and open upon irradiation and thereby reversibly change from a) hydrophobic colored to hydrophilic colorless b) hydrophobic colorless to hydrophilic colored c) hydrophilic colored to hydrophobic colorless or d) hydrophilic colorless to hydrophobic colored. In some embodiments, the moieties can ring close and open upon irradiation to reversibly change from hydrophobic to hydrophilic and from hydrophilic to hydrophobic, without undergoing a change in color.

Thermo-responsive block copolymers display a miscibility gap resulting in an upper or lower critical solution temperature which causes the change in behavior. Ions or zwitterions forming on the surfaces due to these stimuli can also influence the pH and thereby pH induced motions can be triggered on the surfaces. The functionality of some embodiments described herein may be achieved by designing stimuli-sensitive molecular junctions and incorporating these moieties to make polymeric systems.

Synthetic polypeptides or polylactones have several features that make them very attractive for biological applications including low toxicity, biodegradability, predictable structures, and well-controlled dimensions. Synthetic polypeptides or polylactones have been synthesized using a well-studied ring-opening polymerization (ROP) which utilizes a wide variety of monomers containing various functional groups. In particular, carboxylic acid (e.g., glutamate and aspartate) and amino (e.g., lysine) groups of the amino acids have been used to add chemical moieties such as pharmaceutical drugs and molecules that dictate hydrophobicity or pH responsiveness.

Among all the stimuli-responsive polymers, polypeptides are particularly attractive due to their biocompatibility and biodegradability. Another attractive feature of polypeptides is their diverse functionality considering their abundant monomer sources from more than twenty natural amino acids and their synthetic derivatives. Moreover, compared to conventional polymers, polypeptides can form higher ordered secondary structures such as the α-helix and the β-sheet, and the conformational transition among different secondary structures can be triggered by external stimuli. Such features may provide an additional pathway to modulate the stimuli-responsive properties of polypeptides.

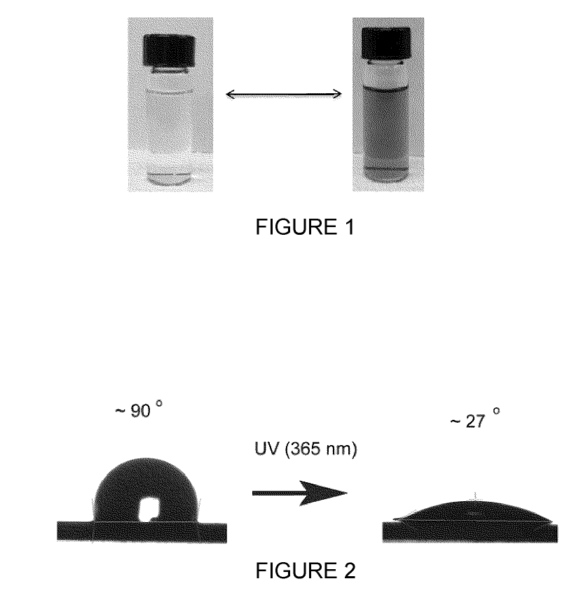

The inventors have designed different stimuli-sensitive (i.e. switchable) monomers as well as their derivatives capable of forming polymers by controlled radical polymerizations like ATRP, NMP, RAFT, and ring opening polymerizations like ROMP, NCA and amino acid synthesis. Incorporating these responsive moieties adds stimuli-responsive functionality to the polymers. FIG. 1 shows an example of the color change associated with a solution of a monomer having a napthacene-based stimuli-switchable moiety upon irradiation with light. The inventors have investigated polymeric brushes as an exemplary system incorporating stimuli-switchable moieties.

Polymeric brushes can provide surfaces that can exhibit stimuli-switchable properties, including photo-switching properties in some example embodiments. In one example embodiment, such surfaces exhibit reversible hydrophobic-hydrophilic, polar-nonpolar, color-colorless etc. changes, which can find many applications in microfluidic and display devices. In one example, tweaking this reversible hydrophobic to hydrophilic change on a particular area of irradiation can enable the movement of water to a specific target and using this water as a carrier, e.g. to deposit a carried molecule at the target location, which has potential application in a microfluidic fabrication system that does not require tedious lithography. Monomers having a photochromic unit which has the capability to change color upon irradiation can be good candidates for making polymer brushes for display devices. This allows, for example, scribbling on the surface using a particular intense visible light source and erasing with a reversible stimuli such as UV or heat.

In some embodiments, activation of the stimuli-switchable moiety results in an irreversible cleavage. In some example embodiments, an irreversibly cleavable surface provides a one-time change in physical property that can be utilized for lithographic applications. The area functionalized by irradiation can have further tailor-made synthetic modifications for patterning. The change in the hydrophobicity observed by the inventors during initial trials is represented in FIG. 2 for an exemplary surface bearing a napthacene-based stimuli-switchable moiety in accordance with one example embodiment. Devices utilizing these surfaces can be promising as they use light as a primary source to switch the properties of the stimuli-switchable moiety, which is considered as abundantly available and an environmentally gracious stimuli.

In one example embodiment, the switchable polymers according to the present disclosure may be cross-linked (e.g. via a functional group) to another protein structure to regulate that protein (e.g. switching the switchable polymers from hydrophilic to hydrophobic could be used to alter the binding of a bound protein construct with its target, thereby turning the bound protein on or off). In alternative embodiments, the switchable polymers may be coupled to a membrane surface and used to turn the membrane on or off. For example, in the case of a membrane used to purify water, coupling switchable polymers to the membrane and switching the polymers between their hydrophilic and hydrophobic states may be used to turn the membrane “on” (i.e. permitting the flow of water therethrough) and “off” (e.g. preventing the flow of water therethrough). In such an embodiment, the switchable polymers may be switchable, for example from hydrophobic to hydrophilic or vice versa, in response to an external stimulus (e.g., light), and therefore affect the physical and/or chemical properties of the associated membrane or proteins.

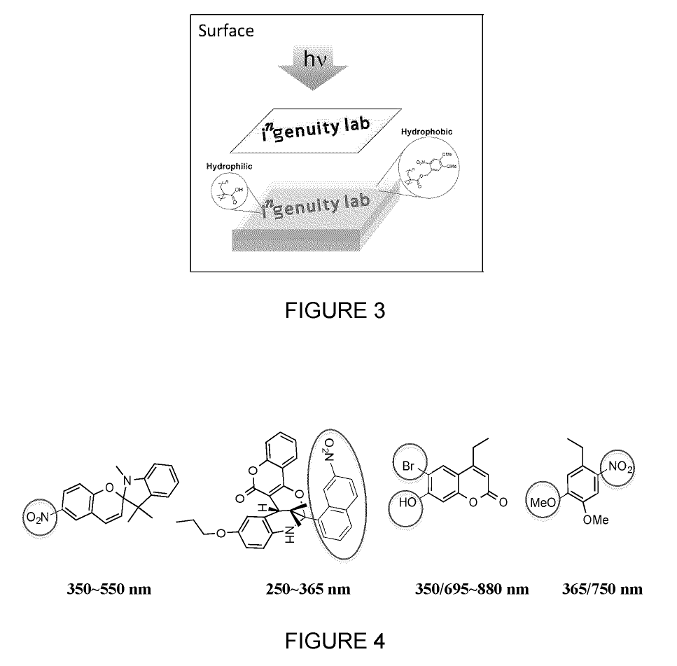

In another example embodiment, the switchable polymers according to some embodiments of the present invention are attached to a substrate surface (e.g., the surface of a microfluidic device, or some other substrate). A photo-mask may be placed above the substrate surface, and the switchable polymers may be switchable, for example from hydrophobic to hydrophilic or vice versa, in response to an external stimulus (e.g., light). As schematically illustrated in FIG. 3, the photo-mask has a particular pattern in the text of “ingenuity lab”. After light illumination, the portion of the substrate surface under the pattern “ingenuity lab” is turned to hydrophilic (colored), while the rest of the substrate surface remains hydrophobic (colorless).

Overall, the combination of structural design and stimulus strategies can bring numerous approaches for solving numerous scientific issues by providing versatile molecular machines and synthetic nanoreactors.

In some embodiments, the stimuli switchable moiety is a photo-responsive moiety, for example, a spiropyran, spirooxazine, coumarin, nitrobenzene, azobenzene, bisthienylethene, flugide, 2-diazo-1,2-napthoquinone-5-sulfonyl-methylacrylamide, napthacene derivative, or the like.

In some embodiments, the stimuli-switchable moiety is a pH-responsive moiety, for example, a napthacene derivative or spiropyran.

In some embodiments, the stimuli-switchable moiety is a temperature-responsive moiety, for example, a napthacene derivative, an azobenzene, or spiropyran.

In some embodiments, the stimuli-switchable moiety is a redox-responsive moiety (e.g. sensitive to-disulfides, peroxides), is responsive to glucose (e.g. boronic acid), or is an enzyme (e.g. which can be modulated by peptides, DNA, RNA, or a particular solute).

Where a molecule incorporates a stimuli switchable moiety that has been previously characterized, it would be within the expected ability of a person of ordinary skill in the art to determine appropriate environmental conditions (e.g. wavelength of light, change in pH, change in temperature) required to switch that stimuli switchable moiety.

In some embodiments, the stimuli switchable moiety is incorporated into or onto a polymeric surface. In some embodiments, the stimuli switchable moiety is incorporated into or onto a polymeric surface to produce a biocompatible surface. A biocompatible surface can, for example, be used to carry out cell attachments or enzymatic studies.

In some embodiments, the stimuli switchable moiety is incorporated into a monomer to produce a stimuli-switchable monomer. In some embodiments, the stimuli-switchable monomers are polymerized together to form a stimuli-switchable polymer. In some embodiments, this process is described as a “pre-polymerization” approach to the synthesis of stimuli-switchable polymers.

In some embodiments, the stimuli switchable moiety is incorporated into a polymer (e.g. after polymerization of suitable monomers to form the polymer). In some embodiments, this process is described as a “post-polymerization” approach to the synthesis of stimuli-switchable polymers.

In some embodiments, the stimuli-switchable monomers are lactone-based monomers, N-carboxyanhydride monomers, or modified amino acids. In some embodiments, the stimuli-switchable monomers are combined in any appropriate manner to produce a stimuli-switchable polymer. In some embodiments, the stimuli-switchable monomers are modified amino acids, and the modified amino acids are incorporated into a peptide at any desired location in the peptide sequence using conventional peptide synthesis techniques. In some such embodiments, the peptide synthesis is conducted directly on a desired surface (for example, as may be useful in a desired application), rather than synthesizing a peptide on a resin conventionally used for peptide synthesis.

In some embodiments, the stimuli-switchable moiety is a napthacene derivative.

In one example embodiment, the stimuli-switchable moiety is based on the structure of napthacene and has one of the general chemical structures (21), (22) or (23) set forth below:

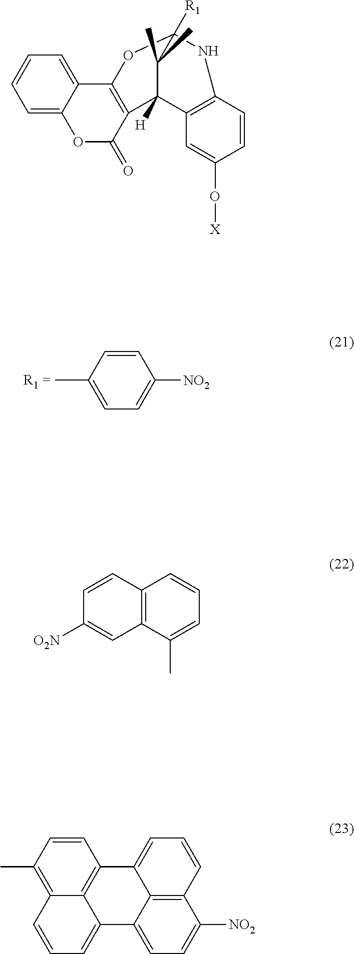

wherein R₁ can be any one of the three groups shown in the right-hand column above, and X denotes the rest of the molecule to which the stimuli-switchable moiety is attached.

In some embodiments having the general structure (21), (22) or (23), the molecule is a monomer that can be used to synthesize a polymer, and in such embodiments X is a functional group that can be used under appropriate reaction conditions to couple the napthacene-based stimuli-switchable moiety to another molecule and/or to polymerize a plurality of monomers having the general structure (21), (22) or (23). In some such example embodiments, X has one of the structures shown below:

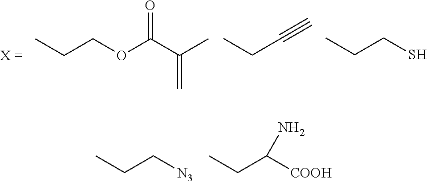

In one example embodiment, a stimuli-switchable moiety having the formula (22) or a molecule incorporating a stimuli-switchable moiety having the formula (22) is provided:

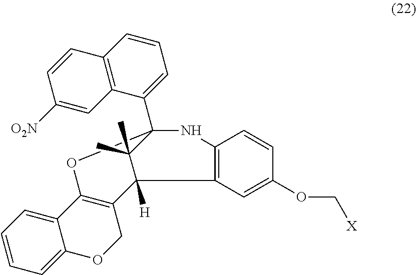

wherein X denotes the rest of the molecule to which the stimuli-switchable moiety is attached. In some such embodiments, the stimuli-switchable moiety is part of a monomer suitable for coupling the stimuli-switchable moiety to another molecule or for polymerization. In some such embodiments, the stimuli-switchable moiety has the structure (22), in which X is a to methylacrylate (24), propargyl (25), thiol (26), azide (27) or glutamic acid (28) moiety.

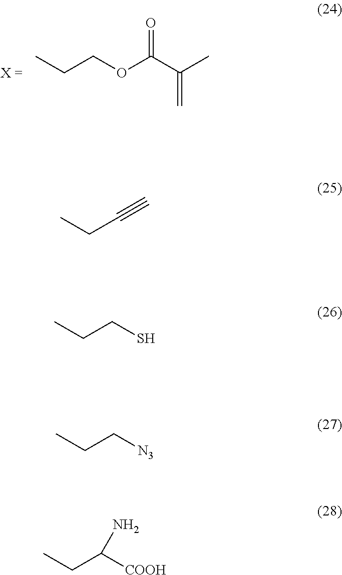

In one example embodiment, a stimuli-switchable moiety having the general formula (21), (22) or (23) is switchable in response to light (i.e. the stimuli-switchable moiety having the formula (21), (22) or (23) is a photoswitchable moiety). Upon irradiation with light having a wavelength of approximately 365 nm or approximately 730 nm, the stimuli-switchable moiety having the formula (21), (22) or (23) reversibly switches from a colorless/hydrophobic state to a purple coloured/hydrophilic state, as shown in FIG. 1 and Scheme 3. The hydrophilic state includes a zwitterionic charge (i.e. while the overall charge of the moiety is neutral, one positive and one negative charge are present within the moiety). It will be apparent to those skilled in the art that the wavelength of light used to switch the stimuli-switchable moiety having the formula (21), (22) or (23) need not be precisely 365 nm or 730 nm, and that similar wavelengths of light could be used, e.g. in some embodiments light having a wavelength between about 250 nm and about 450 nm, including any value therebetween, e.g. 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440 or 445 nm could be used; or light having a wavelength between about 650 nm and 800 nm, including any value therebetween, e.g. 655, 670, 675, 680, 685, 690, 695, 700, 705, 710, 715, 720, 725, 730, 735, 740, 745, 750, 755, 760, 765, 770, 775, 780, 785, 790 or 795 nm could be used. In some embodiments, by modifying the structure of the stimuli-switchable moiety, the wavelength of light that can be used to switch the moiety can be adjusted.

The wavelength used to switch light-responsive switchable moieties is based on absorption of light of a particular wavelength by the chromophore of the photoswitchable moiety. For example, in spiropyran or naphthacene, the moiety photochemically cycles from its closed form, in which it primarily absorbs light in the UV region, to its open form, which has a visible absorption band due to its extensively conjugated π-electron cloud. Also spiropyran or naphthacene are practically colorless, since the lowest electronic transition takes place in the region of the near-ultraviolet (λ<400 nm). This excitation produces the break of the C—O bond in an excited singlet state in a few picoseconds. The subsequent rotation across the C—C bond leads to an open photoisomer, which absorbs strongly in the visible region (λ=500˜700 nm) because of the π-electronic delocalization by conjugation. As opposed to the closed forms, these photoisomers show an intense coloration and a high dipole moment. The substitution of electron withdrawing or electron donating groups, such as —OMe, —NO₂, —F, —COOH, —C≡N, has strong influence in tuning the absorption wavelength of the system. Thus, by making appropriate structural modifications to the photoswitchable moiety, the wavelength of electromagnetic radiation used to switch the photoswitchable moiety can be adjusted.

As shown in FIG. 4, in which electron-withdrawing or electron-donating groups are circled, spiropyran can be switched from one configuration to the other by application of light having a wavelength in the range of about 350 nm to about 550 nm; an exemplary napthacene-based photochromic moiety can be switched from one configuration to the other by application of light having a wavelength in the range of about 250 nm to about 365 nm; a coumarin-based photochromic moiety can be switched from one configuration to the other by application of light having a wavelength in the range of about 350 nm, or in the range of about 695 nm to about 880 nm; and a nitro-based photoswitchable moiety can be switched from one configuration to the other by application of light having a wavelength in the range of about 365 nm or about 750 nm. Other photoswitchable moieties and the wavelength of light used to switch that particular moiety are known in the art and could be used in some embodiments of the present invention.

In one example embodiment, a napthacene-based stimuli-switchable moiety is switchable in response to heat. In one example embodiment, a stimuli-switchable moiety having the general structure (21), (22) or (23) is switchable in response to heat. For example, in some embodiments, increasing the temperature to a temperature greater than approximately 50° C. results in a conformational change of a stimuli-switchable moiety having the general structure (21), (22) or (23) from the hydrophilic state to the hydrophobic state.

In one example embodiment, a stimuli-switchable moiety having the general structure (21), (22) or (23) is switchable from a hydrophobic configuration to a hydrophilic configuration upon the application of light having a wavelength in the range of about 300 nm to about 750 nm (for example, light having a wavelength between about 250 nm and about 450 nm, including any value therebetween, e.g. 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440 or 445 nm; or light having a wavelength between about 650 nm and 800 nm, including any value therebetween, e.g. 655, 670, 675, 680, 685, 690, 695, 700, 705, 710, 715, 720, 725, 730, 735, 740, 745, 750, 755, 760, 765, 770, 775, 780, 785, 790 or 795 nm), and is switchable from the hydrophilic configuration to the hydrophobic configuration by either the application of light having a wavelength in the range of about 300 nm to about 750 nm (for example, light having a wavelength between about 250 nm and about 450 nm, including any value therebetween, e.g. 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440 or 445 nm; or light having a wavelength between about 650 nm and 800 nm, including any value therebetween, e.g. 655, 670, 675, 680, 685, 690, 695, 700, 705, 710, 715, 720, 725, 730, 735, 740, 745, 750, 755, 760, 765, 770, 775, 780, 785, 790 or 795 nm), or by the application of heat, as shown in Scheme 3 below for a compound having the general structure (22) in which the hydrophobic configuration has the structure (29) and the hydrophilic configuration has the structure (30). In one such example embodiment, the molecule switches from the hydrophobic configuration to the hydrophilic configuration upon application of light having a wavelength of approximately 365 nm, or of approximately 730 nm. The molecule switches from the hydrophilic configuration to the hydrophobic configuration upon an increase in temperature to above approximately 50° C. In this particular example embodiment, changes in temperature will not cause the hydrophobic configuration to switch to the hydrophilic configuration. However, the hydrophilic configuration can be switched to the hydrophobic configuration by increasing the temperature to approximately 50° C. or higher. Similarly, compounds having the general structure (21) or (23) will not change configuration in response to changes in temperature when in the hydrophobic configuration, but will switch from the hydrophilic configuration to the hydrophobic configuration upon heating.

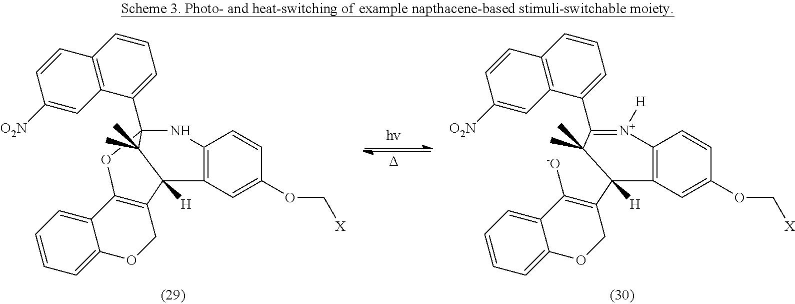

In some embodiments, according to Scheme 3, X:— ═, ≡, SH, N₃.

In one example embodiment, a stimuli-switchable moiety having the general structure (21), (22) or (23) is switchable between a hydrophobic configuration and a hydrophilic configuration in response to changes in pH. Without being bound by theory, it is believed based on the chemical structure of (21), (22) or (23) that the molecule will likely be hydrophilic at low pH, and hydrophobic at high pH.

In some embodiments, an amino acid monomer bearing stimuli-switchable moiety based on napthacene is synthesized by a multi-step organic synthesis. In one example embodiment, a method of synthesizing such a stimuli-switchable moiety coupled to an aspartic acid or glutamic acid amino acid comprises (a) functionalizing 4-hydroxyl benzyl amine to be polymerizable/clickable by the Williamson ether reaction, as described for example in Williamson, 2009. The next step (b) is coupling of the protected amino acid (i.e. aspartic acid or glutamic acid) to a 4-hydroxy benzyl amine by EDC/DMAP coupling, followed by deprotection and reaction with N-Carboxyanhydride to form a monomer for polypeptide synthesis. The next step (c) is reacting the 4-amino phenyl (propargyl/allyl/thio acetate/bromo alkyl/amino acid) with a 4-nitro benzaldehyde/7-nitro naphthaaldehyde/4-nitro 10 perylenecarbaldehyde, by amino alkylation under conditions and for a time sufficient for coupling to occur between the terminal amine and aldehyde compound, thereby yielding an iminium derivative; and then (d) reacting isobutryaldehyde with a compounds as described above, in the presence of a catalytic amount of ytterbium(III) trifluoromethanesulfonate to yield cyclic alcohols. Finally, (e) the reaction of the resultant cyclic alcohol with 4-hydroxycoumarin in the presence of a catalytic amount of p-toluenesulfonic acid under reflux conditions.

In some embodiments, acrylic monomers incorporating a stimuli-switchable moiety are prepared. For example, in one example embodiment a methacrylate monomer bearing a stimuli-switchable moiety having the structure (21), (22) or (23) is prepared according to Scheme 4 starting from compound (34) obtained according to Scheme 5. Such acrylic monomers are suitable for polymerization to form a polymer via radical polymerization. In some embodiments, the radical polymerization can be carried out using atom transfer radical polymerization (ATRP).

In some embodiments in which it is desired to produce a biocompatible surface, the radical polymerization can be carried out by nitroxide-mediated polymerization (NMP) or by reversible addition fragmentation polymerization (RAFT).

In one example embodiment, an monomer having an alkyne functional group incorporating a stimuli-switchable moiety having the chemical structure (22) wherein X comprises propargyl (referred to as structure (25)) is provided. In one example embodiment, a compound having the structure (25) is synthesized according to the general reaction shown in Scheme 4 below.

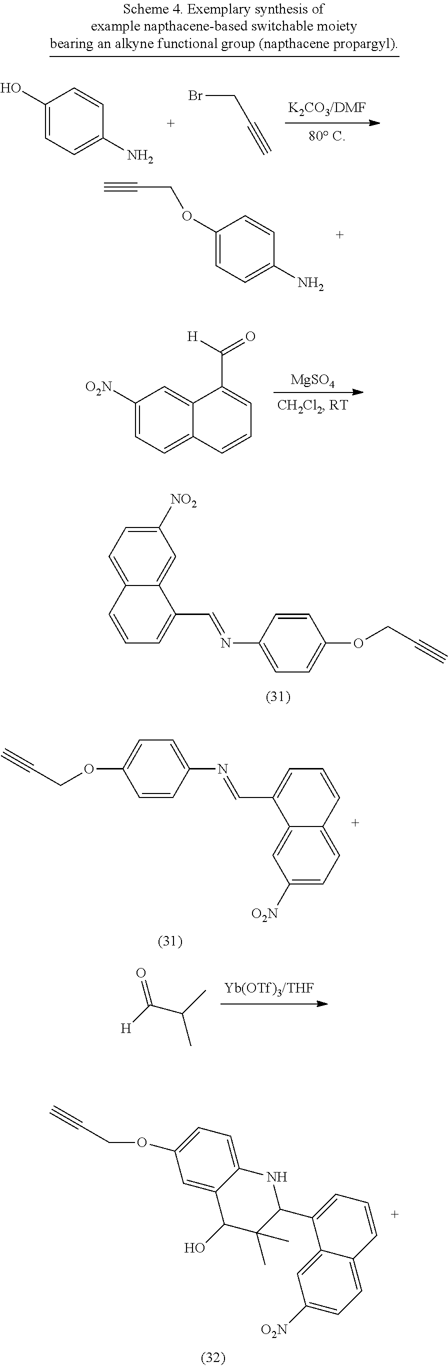

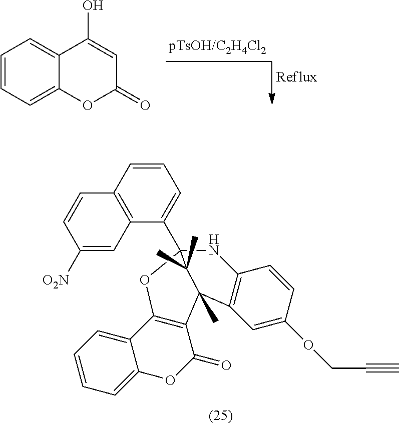

Other monomers incorporating a napthacene-based stimuli-switchable moiety, including a napthacene-based stimuli-switchable moiety having the structure (21), (22) or (23) can be synthesized from appropriate starting materials following a similar reaction scheme. Suitable starting materials are shown below in Scheme 5. Compounds (33) and (34) can be used to synthesize polymerizable monomers incorporating a napthacene-based stimuli switchable moiety. Compounds (35) and (36) can be used to synthesize clickable monomers incorporating a napthacene-based stimuli switchable moiety (i.e. monomers suitable for post-polymerization modification of a polymer using click chemistry). Compound (37) can be used to synthesize an amino acid (glutamic acid in this example) incorporating a napthacene-based stimuli switchable moiety, which can be used in direct peptide synthesis, or alternatively deprotected and converted to its corresponding N-carboxyanhydride monomer for polymerization to generate a polymer incorporating a napthacene-based stimuli switchable moiety.

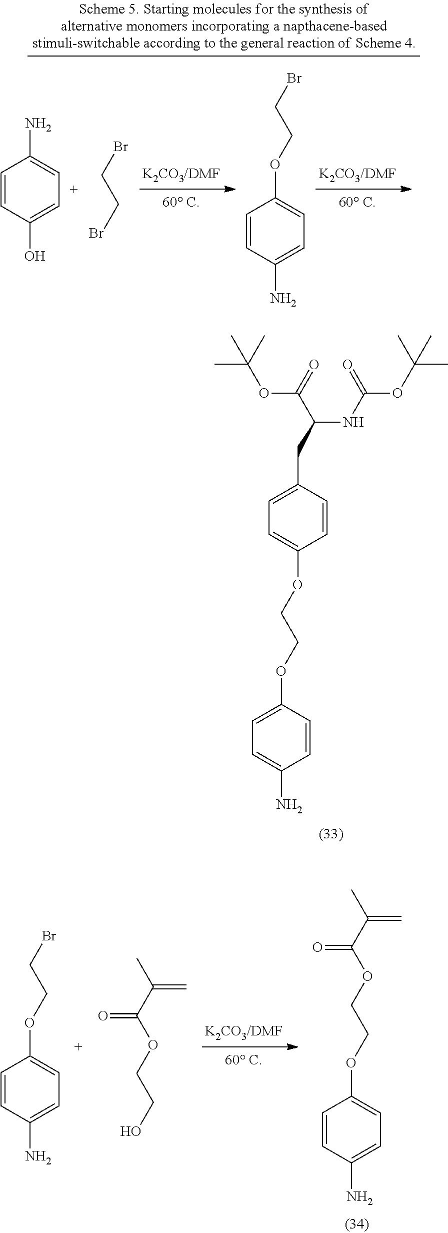

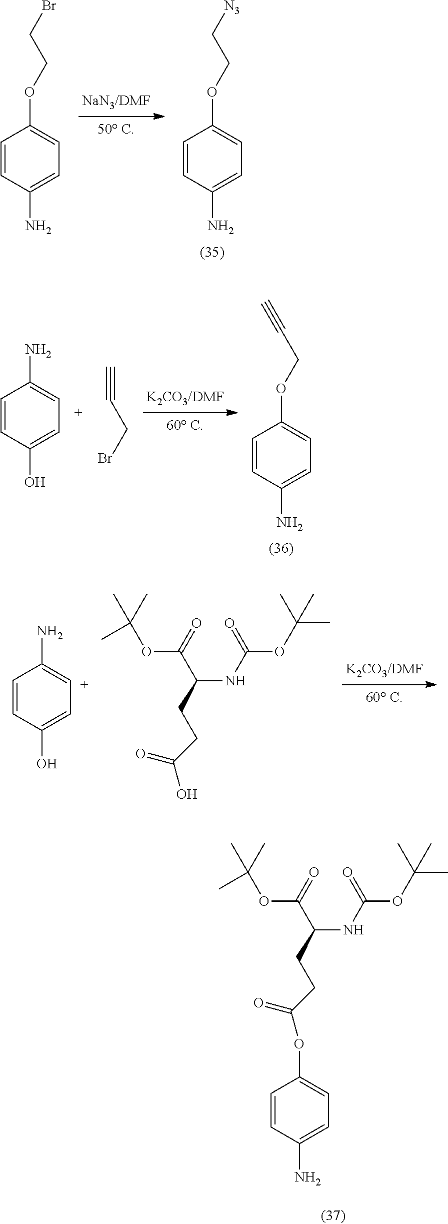

In some embodiments, polymers incorporating a napthacene-based stimuli-switchable moiety, including for example polymers incorporating stimuli-switchable moieties having the general structure (21), (22) or (23) are used in a microfluidic chip, a display device, a selective size separator, a transducer, a nanoparticle, a membrane, for particle and/or droplet manipulation, for the construction of self-cleaning surfaces, anti-biofouling surfaces, anticorrosive surfaces, actuators, chemical sensors, photolithography, or holographic data storage.

In one example embodiment, monomer having an alkyne functional group having the structure (25) is incorporated into a surface prepared using atom transfer radical polymerization (ATRP) according to the general process set forth in Scheme 6 below. Scheme 6 illustrates a post-polymerization modification of a polymer to incorporate a stimuli-switchable moiety. In Scheme 6, post functionalization of azide terminated ATRP polymers with propargyl terminated napthacene having the structure (25) is carried out. More specifically, glycidyl methacrylate is polymerized using ATRP and then post functionalized using a light-responsive monomer containing a complementary clickable group to facilitate coupling of the monomer to the polymer. In some embodiments, this post-polymerization approach can be used for fabrication of complex polymer brush architectures, for example where it is desired to have the stimuli-switchable moiety only on the surface of the polymer, as opposed to distributed within the bulk of the polymer matrix, as would occur for polymers made by the direct polymerization of monomers bearing stimuli-switchable moieties. In alternative embodiments, direct atom transfer radical polymerization (ATRP) of acrylic naphathacene can be carried out using established methods.

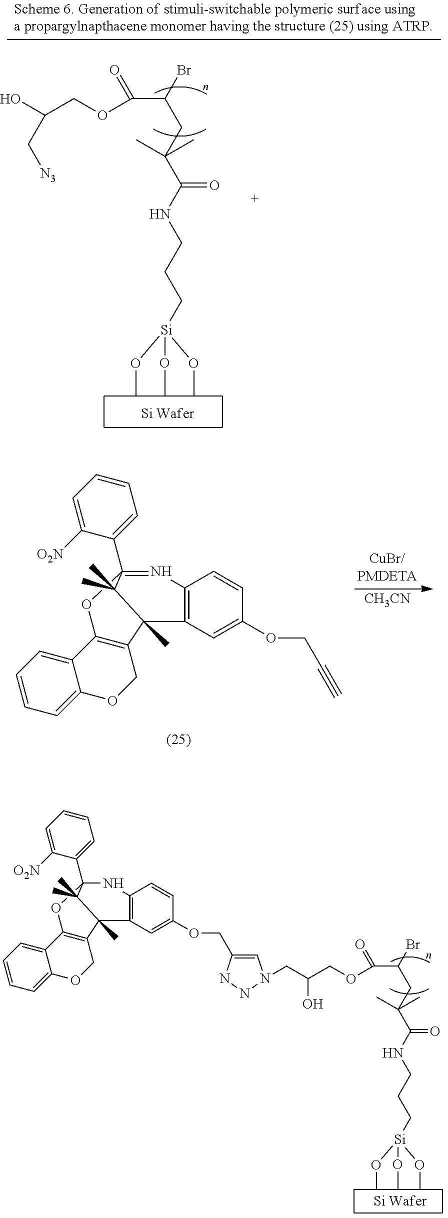

In one example embodiment, a napthacene methyacrylate monomer having the structure (24) (i.e. structure (22) wherein X=methacrylate) is directly incorporated into a surface using surface-initiated ATRP according to the general process set forth in Scheme 7 below. In alternative embodiments, a propargyl napthacene monomer having the structure (25) could be directly incorporated into a surface using surface-initiated ATRP according to Scheme 7, rather than by using the post-modification approach of Scheme 6.

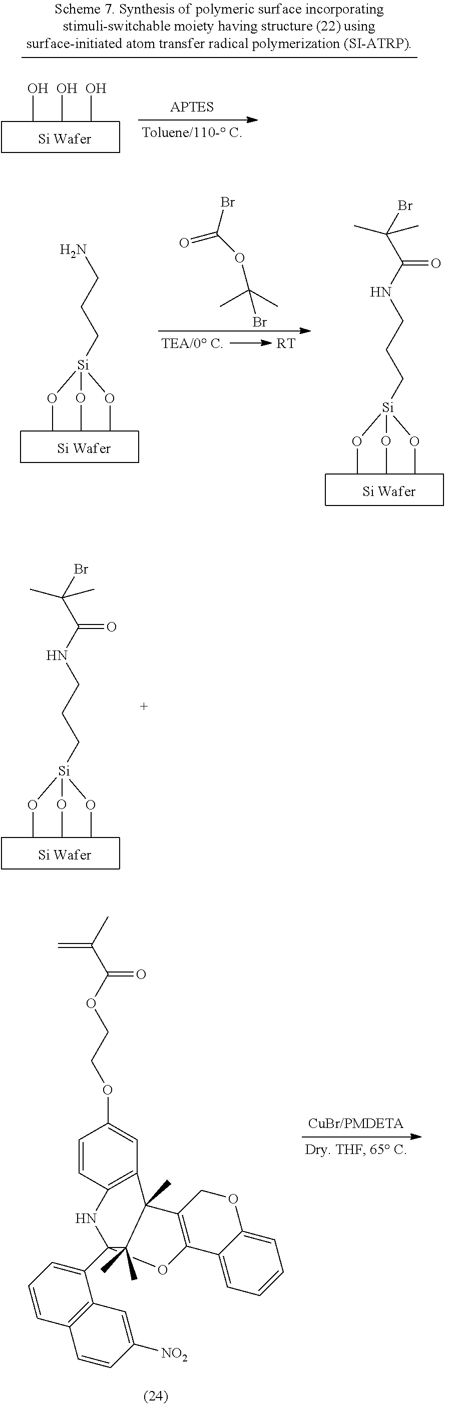

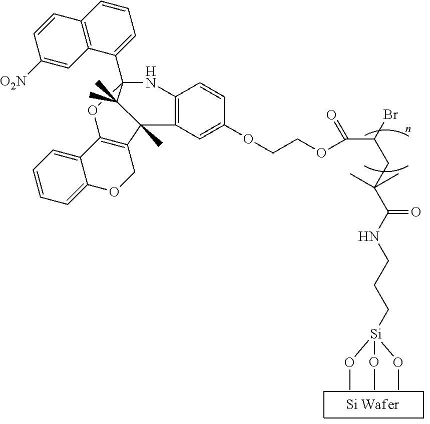

In some embodiments, monomers incorporating a stimuli-switchable moiety are prepared, and the monomers are polymerized together using any suitable technique in order to produce a polymer incorporating the stimuli-switchable moiety. In some embodiments, this is referred to as a pre-polymerization modification approach.

In alternative embodiments, a stimuli-switchable moiety, including a napthacene-based stimuli-switchable moiety, for example having the structure (21), (22) or (23) can be incorporated into a polymer using a post-polymerization approach using thiolene or epoxyamine chemistry, which would be within the expected ability of the person of ordinary skill in the art to carry out.

In some embodiments, a polymeric surface bearing or incorporating a napthacene-based stimuli-switchable moiety having the structure (21), (22) or (23) provides a very hydrophobic surface that can be reversibly switched to a hydrophilic surface. The inventors have demonstrated that a polymeric surface bearing a stimuli-switchable moiety having the structure (22) exhibits a change in hydrophobicity (as measured by the contact angle, θ_{adv}) that is approximately 1.5 times greater than the change in hydrophobicity (as measured by the contact angle, θ_{adv}) exhibited by a corresponding polymeric surface incorporating a nitrobenzene-derived stimuli-switchable moiety when the stimuli-switchable moiety is switched from its hydrophobic configuration to its hydrophilic configuration. A strong increase in hydrophobicity of the stimuli-switchable moiety may be advantageous in certain applications, for example micropatterning, selective adhesion, microfluidics, and the like.

In some embodiments, the monomers are polymerized on a suitable starting material to produce a polymeric surface. Depending on the method to be used to synthesize the polymer, an appropriate starting material for polymer synthesis, referred to as an initiator, is prepared. Example reaction schemes for producing exemplary initiators for polymer synthesis using ring-opening polymerization (RO) or N-carbooxyanhydride chemistry (NCA), atom transfer radical polymerization (ATRP), reversible addition fragmentation polymerization (RAFT), nitroxide-mediated polymerization (NMP), or click chemistry, respectively, are shown in Scheme 8 below.

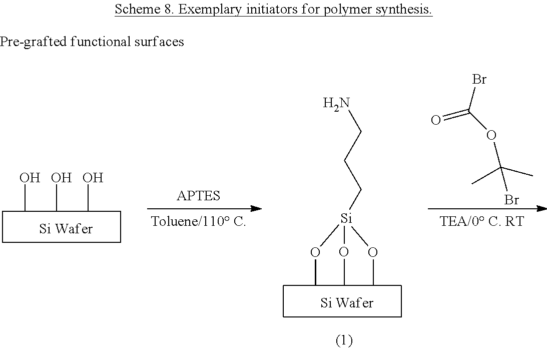

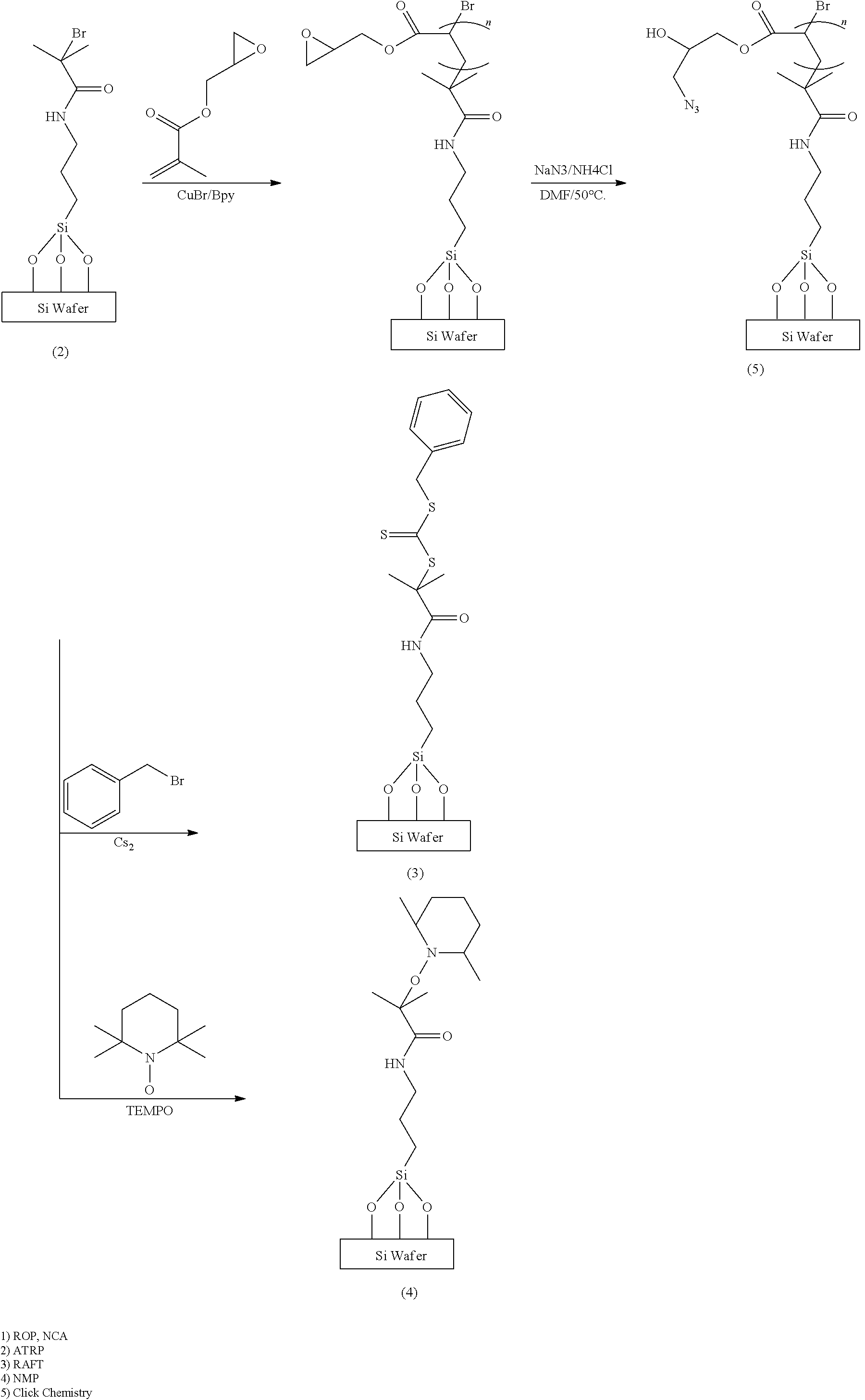

A further example of an initiator that can be used to carry out polymer synthesis using ATRP is shown in Scheme 9.

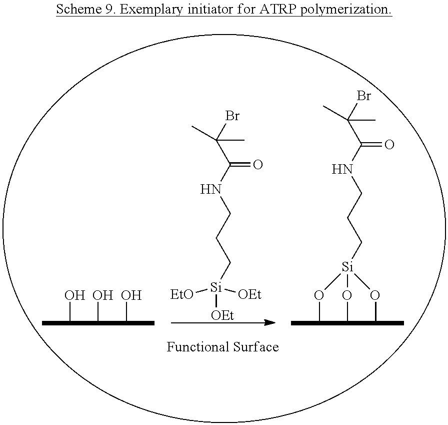

In some embodiments, monomers incorporating stimuli-switchable moieties for carrying out ring opening polymerization are provided. In some embodiments, the monomers are lactone-based monomers. In some embodiments, the monomers are caprolactone-based monomers or nonalactone-based monomers.

In some embodiments, the nonalactone-based monomers have the general chemical structure (49) and the caprolactone-based monomers have the general chemical structure (50) shown below:

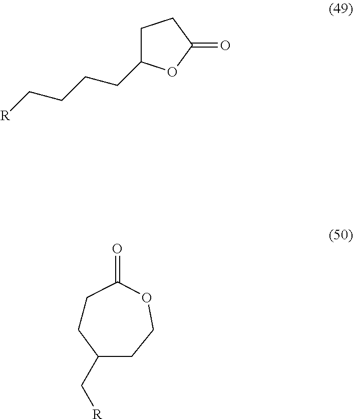

wherein R denotes a stimuli-switchable moiety. R can be any suitable stimuli-switchable moiety, for example a spiropyran, spirooxazine, coumarin, nitrobenzene, azobenzene, bisthienylethene, flugide, 2-diazo-1,2-napthoquinone-5-sulfonyl-methylacrylamide, napthacene derivative, or the like, including a napthacene-based stimuli-switchable moiety having the general structure (21), (22), or (23), including for example the structures (51), (52), (53) or (54) shown below incorporating nitrobenzyl, napthacene-based, coumarin and spiropyran stimuli-switchable moieties, respectively. The stimuli-switchable moiety can be provided at any suitable location on the lactone ring. Additional substituents can be present on the lactone ring.

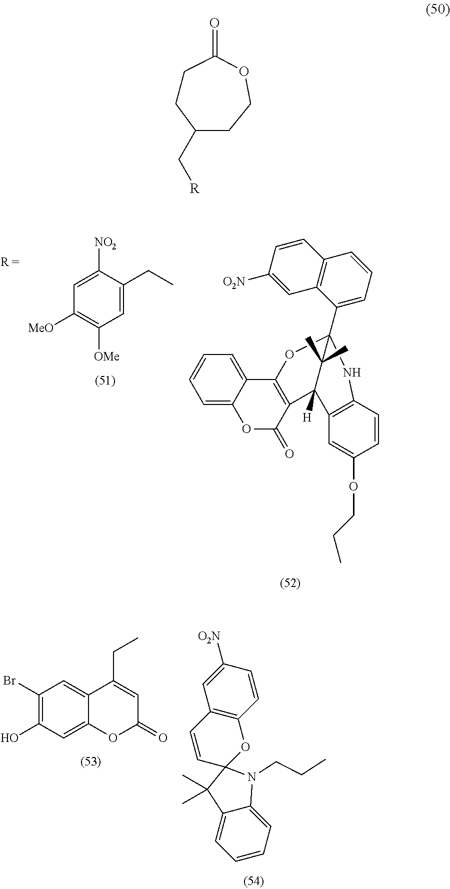

In some embodiments, the lactone-based monomers are based on any suitable lactone, for example, butyrolactone, valerolactone, caprolactone, nonalactone, or the like. In some embodiments, the lactone-based monomers are based on a lactone having any one of the following structures (e.g. a 5-membered ring structure (55), a 6-membered ring structure (56), a 7-membered ring structure (57), an 8-membered ring structure (58), a 9-membered ring structure (59), or the like), in which the stimuli-switchable moiety can be provided at any desired location on the ring structure, and with additional substituents optionally present on the carbon atoms of the ring structure.

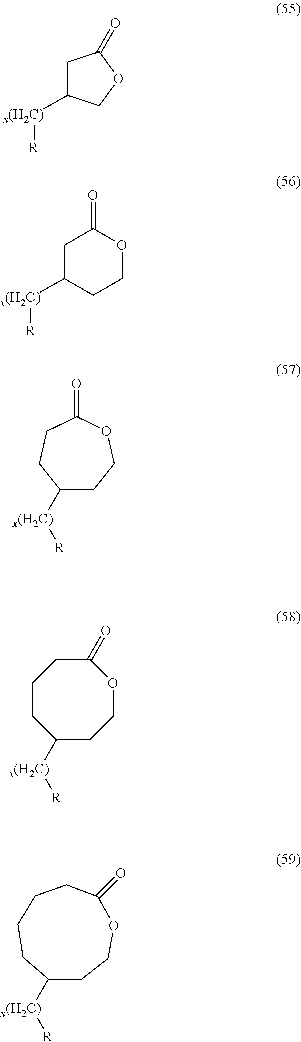

In some embodiments, the lactone-based monomers are synthesized according to the general reaction scheme illustrated in Scheme 10 below.

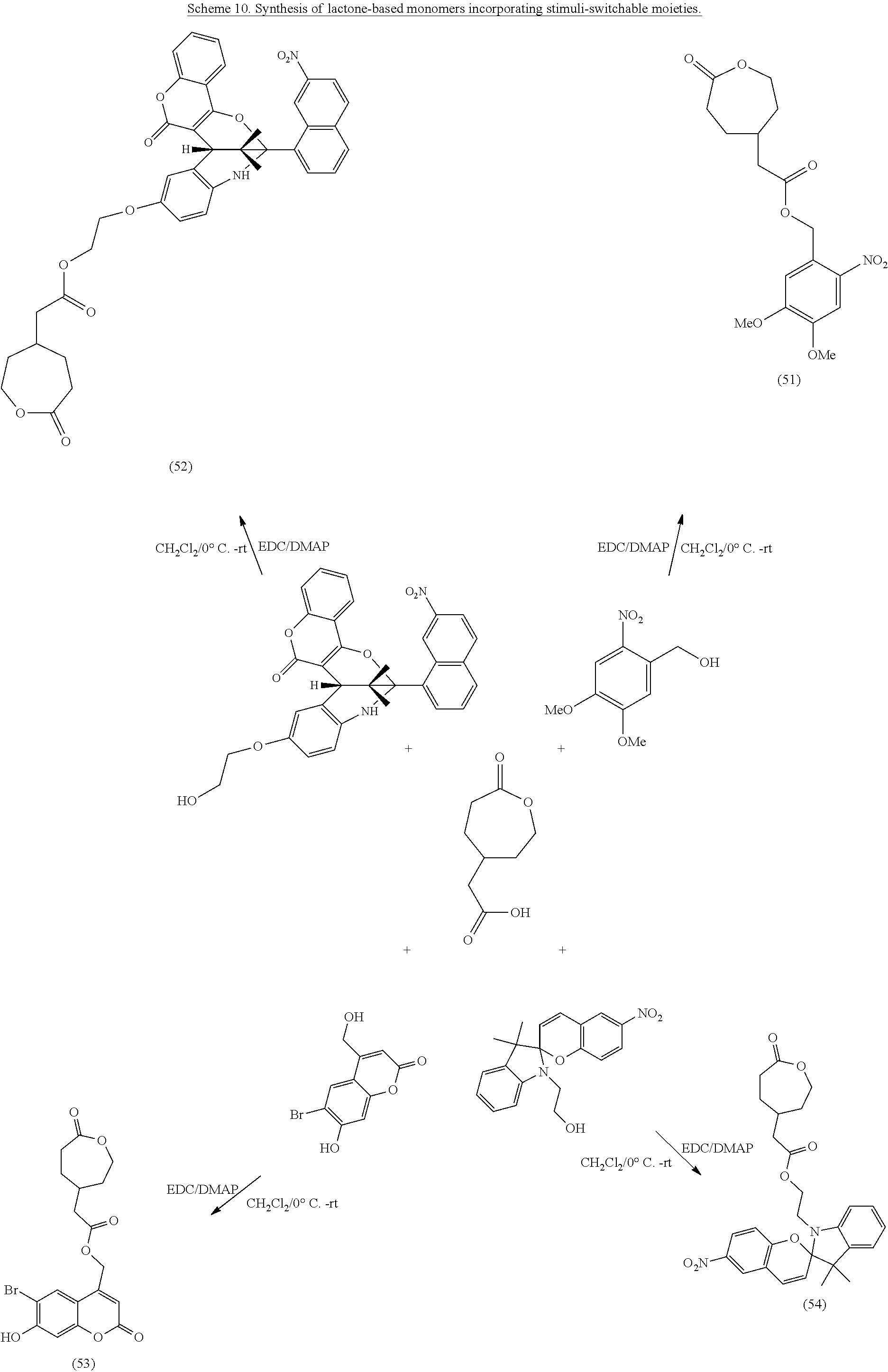

In general, lactone-based monomers incorporating stimuli-switchable moieties can be synthesized by a simple EDC coupling reaction of the appropriate lactone bearing carboxylic acid and stimuli-switchable moieties bearing an alcohol functional group. At first, a lactone bearing a carboxylic acid moiety was synthesized, then carbodiimide (EDC) mediated coupling with the hydroxyl group of the appropriate photo-responsive moiety was carried out to form the corresponding esters. EDC reacts with carboxylic acid groups to form an active O-acylisourea intermediate that is easily displaced by nucleophilic attack from primary alcohol groups in the reaction mixture. The primary alcohol forms an ester bond with the original carboxyl group, and an EDC by-product is released as a precipitated urea derivative. In representative experiments conducted by the inventors, the final product was purified by silica gel column chromatography (EA/Hex 1:1) to afford 65˜96% yield. In alternative embodiments, the lactone-based monomers incorporating a stimuli-switchable moiety are synthesized by Williamson etherification.

In some embodiments, lactone-based monomers incorporating a stimuli-switchable moiety are used to synthesize a polymer incorporating the stimuli-switchable moiety. In some embodiments, polymerization is carried out using ring opening polymerization. In some embodiments, the synthesis is carried out on a surface, and the initiator for the ring opening polymerization is the structure labeled as (1) in Scheme 8. In some embodiments, the initiator for the ring-opening polymerization is that shown in Scheme 11, and the synthesis of the polymer is carried out according to the general steps of Scheme 11. In some embodiments, the synthesis is carried out in solution. The ring opening polymerization can be carried out in any suitable manner known to those skilled in the art, and the example shown in Scheme 11 is merely representative.

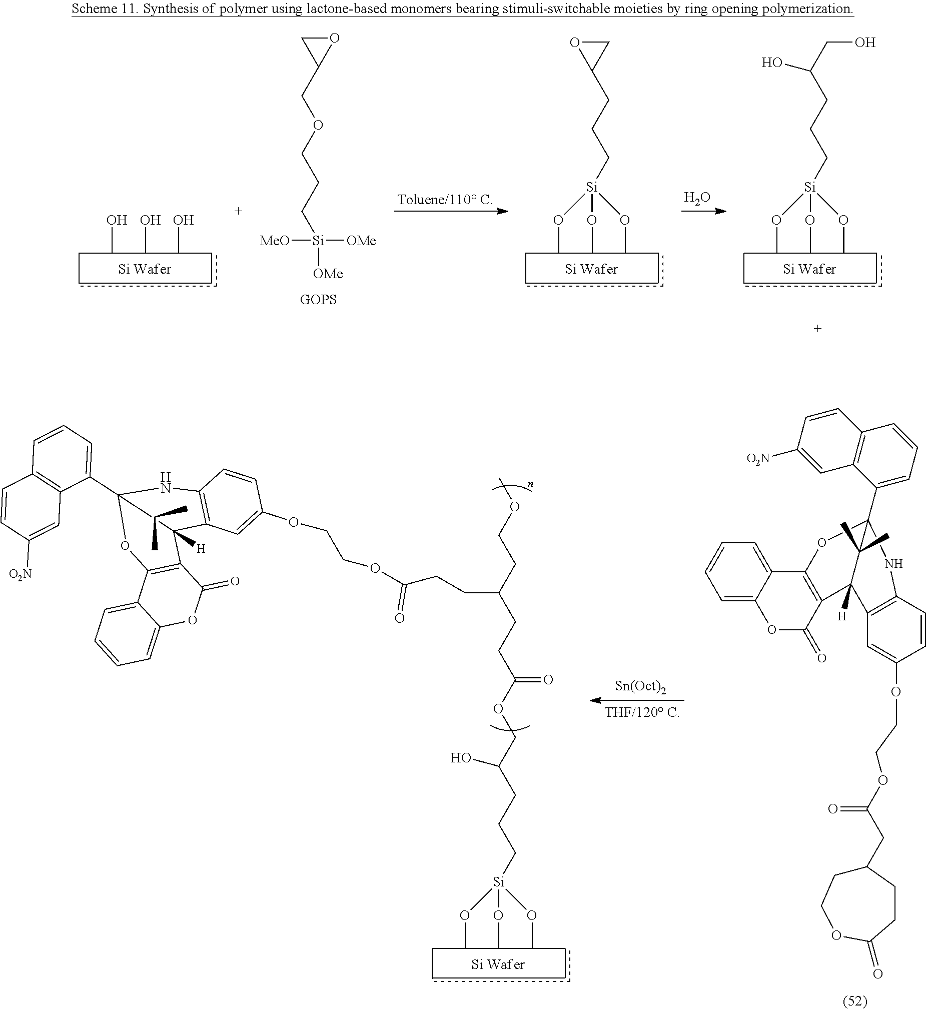

In general, in this example using a silicon surface, polymerization of lactone-based monomers incorporating stimuli-switchable moieties is initiated by the modified/activated hydroxyl groups of a silicon wafer. This modification is carried out by reaction of the 3-(glycidoxypropyl)trimethoxysilane (GOPS) with treated silicon wafer, followed by hydrolysis. These hydroxyl initiators on the surface are used for ring opening polymerization along with stannous octoate as catalyst. The stannous alkoxide active center initiates the ring-opening polymer by consuming the corresponding lactones.

In some embodiments, modified amino acids incorporating a stimuli-switchable moiety are prepared. In one example embodiment, the amino acid incorporating the stimuli-switchable moiety is synthesized using Williamson's synthesis as shown in Scheme 12. In one example embodiment, the amino acid incorporating the stimuli-switchable moiety is synthesized using EDC coupling as shown in Scheme 14. In either embodiment, common amino acids (e.g. protected either by BOC or FMOC chemistry) can be used as a starting material for the synthesis. After modification to incorporate a stimuli-switchable moiety, the protected modified amino acid can be used in peptide synthesis using conventional techniques, modified as may be necessary to avoid conditions that would damage the stimuli-switchable moiety. In further embodiments, common amino acids (e.g. protected either by BOC or FMOC chemistry) can also be used as a starting material for synthesis of a modified amino acid according to Schemes 13 and 15.

In one example embodiment, a protected tyrosine incorporating a stimuli-switchable moiety having the general structure (71) is synthesized according to the general method for Williamson's synthesis shown in Scheme 12, which shows the specific example of a producing a monomer bearing a napthacene-based stimuli-switchable moiety from a commercially available protected tyrosine. In some embodiments, the protected amino acid monomer (70) is used directly for peptide synthesis using conventional techniques. In some embodiments, the protected amino acid monomer (70) is deprotected to yield (71), which can be converted to N-carboxyanydride napthacene-tyrosine having the structure (72) to provide a monomer suitable for polymerization.

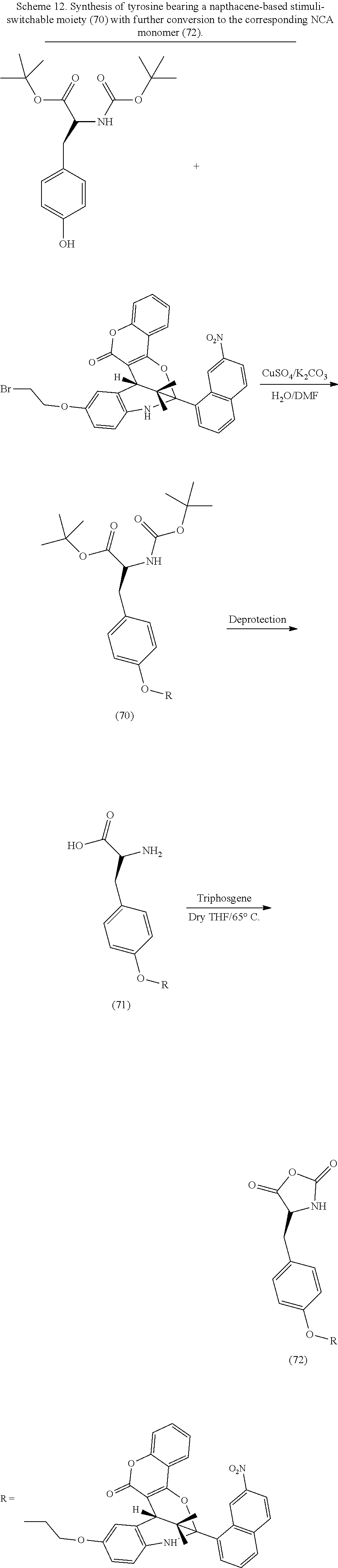

Scheme 13 shows an example scheme for the synthesis of an amino acid, cysteine, incorporating a nitrobenzyl stimuli-switchable moiety (74), with the further conversion to its corresponding N-carboxyanhydride monomer (75), starting from a commercially available protected cysteine. In some embodiments, the protected modified amino acid (73) is used for peptide synthesis using conventional peptide synthesis techniques.

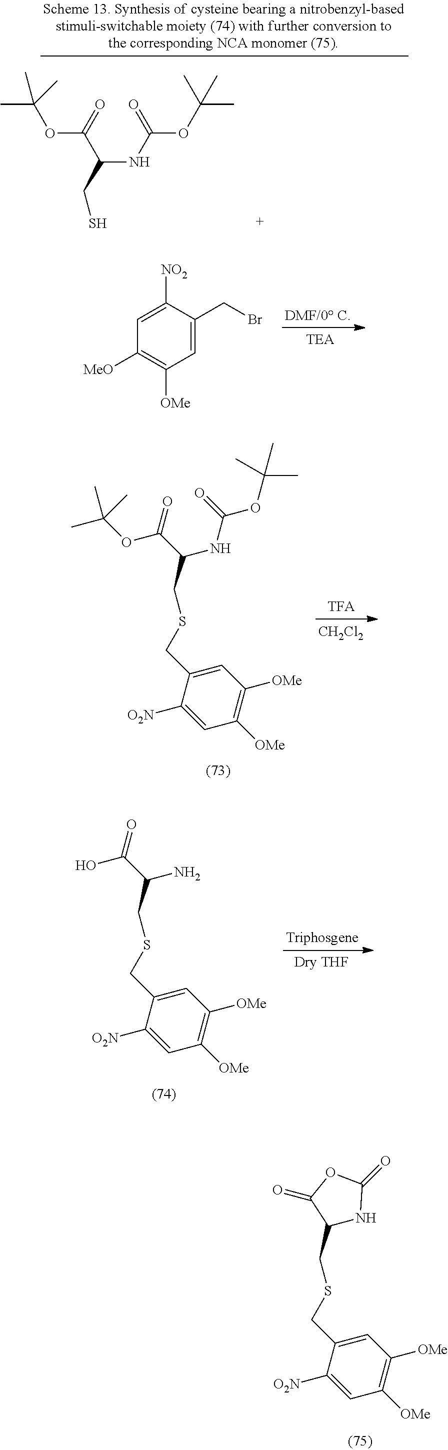

Scheme 14 shows an example scheme for the synthesis of an amino acid, glutamic acid, incorporating a spiropyran stimuli-switchable moiety (77) using EDC coupling, with the further conversion to its corresponding N-carboxyanhydride monomer (78), starting from a commercially available protected amino acid. A similar synthetic route can be used to obtain aspartic acid incorporating a spiropan stimuli-switchable moiety, with the further conversion to its corresponding N-carboxyanhydride monomer. In some embodiments, the protected modified amino acid (76) is incorporated into a peptide using conventional peptide synthesis techniques.

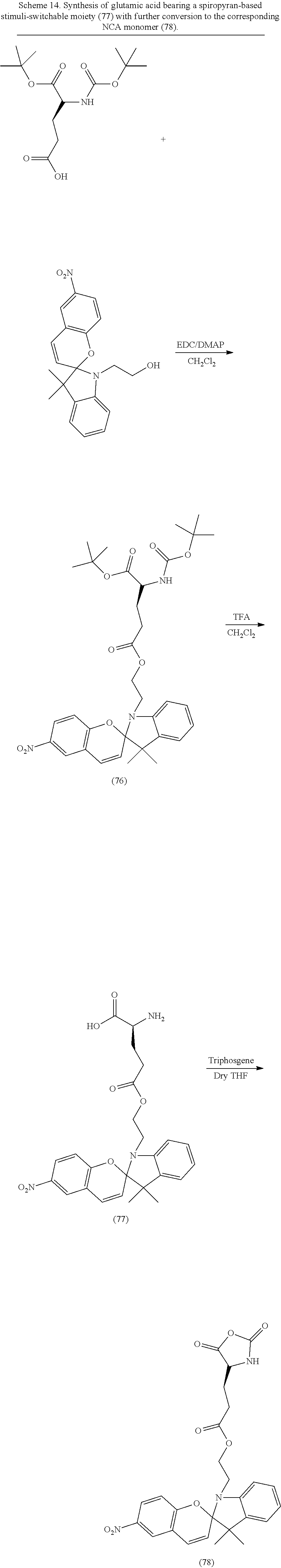

Scheme 15 shows an example scheme for the synthesis of an amino acid, aspartic acid, incorporating a coumarin-based stimuli-switchable moiety (80), with the further conversion to its corresponding N-carboxyanhydride monomer (81). A similar method of synthesis can be used to obtain glutamic acid incorporating a coumarin-based stimuli-switchable moiety, and to further convert that compound to its corresponding N-carboxyanhydride monomer. In some embodiments, the protected modified amino acid (79) is incorporated into a peptide using conventional peptide synthesis techniques.

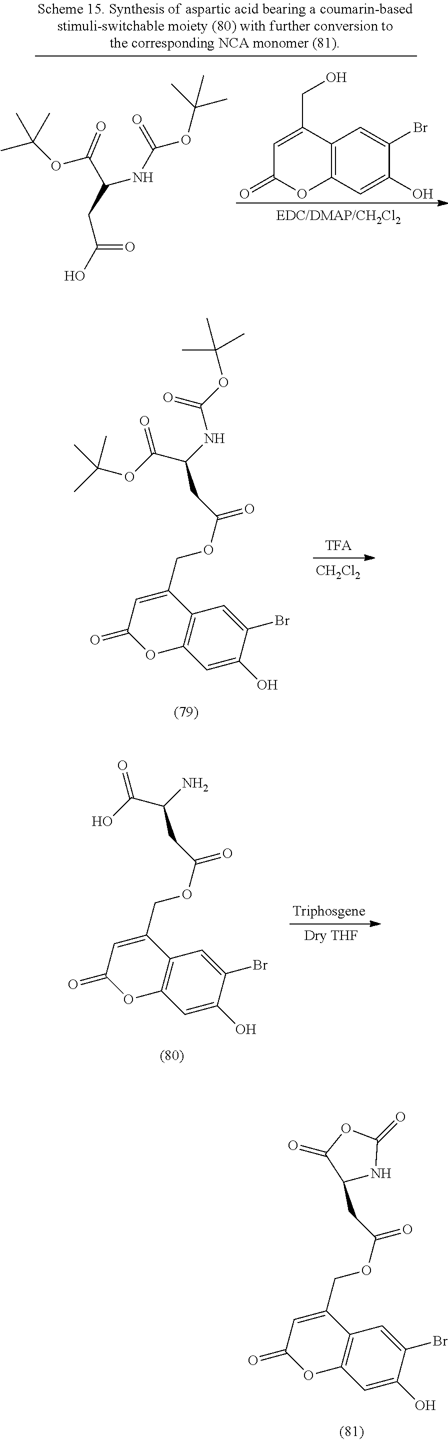

In alternative embodiments, other amino acids can be modified to incorporate stimuli-switchable moieties using similar synthetic techniques. The modified amino acids that can be generated include versions of all twenty naturally occurring amino acids and their stimuli-switchable derivatives, for example as shown in Table 1, as well as modified or non-naturally occurring amino acids and their stimuli-switchable derivatives, for example those non-naturally occurring amino acids shown in Table 2.

TABLE 1
Naturally occurring amino acids that can be modified with
stimuli-switchable moieties.

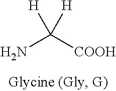

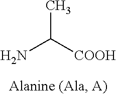

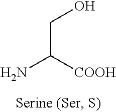

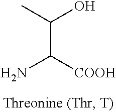

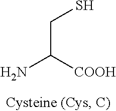

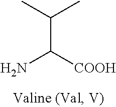

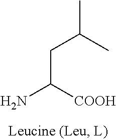

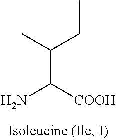

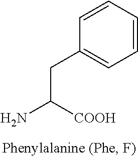 [SMILES: NC(Cc1ccccc1)C(=O)O]

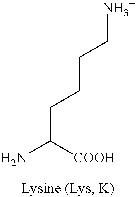

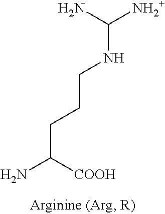 [SMILES: NC([NH3+])NCCCC(N)C(=O)O]

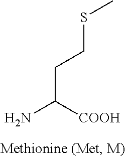

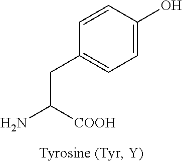 [SMILES: NC(Cc1ccc(O)cc1)C(=O)O]

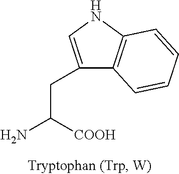 [SMILES: NC(Cc1c[nH]c2ccccc12)C(=O)O]

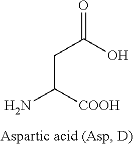

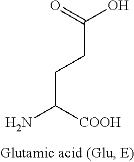

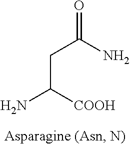

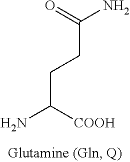

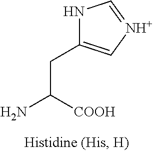 [SMILES: NC(Cc1c[nH+]c[nH]1)C(=O)O]

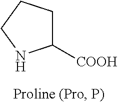

TABLE 2
Exemplary modified amino or non-naturally occurring amino acids that can
incorporate stimuli-switchable moieties.

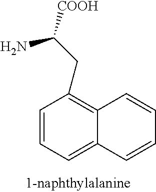

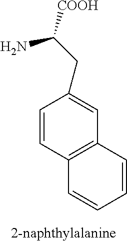

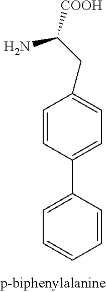

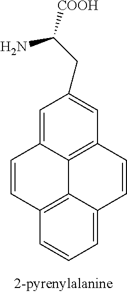

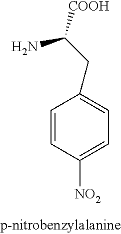

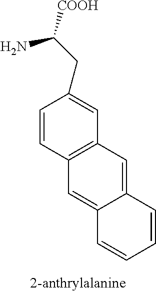

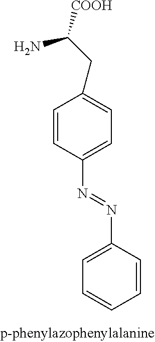

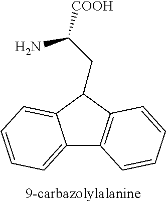

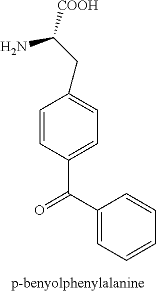

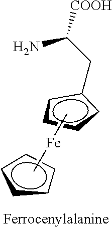

Scheme 16 summarizes some potential synthetic routes to various amino acids incorporating stimuli-switchable moieties. In alternative embodiments, other amino acids can be modified to incorporate stimuli-switchable moieties using similar synthetic techniques.

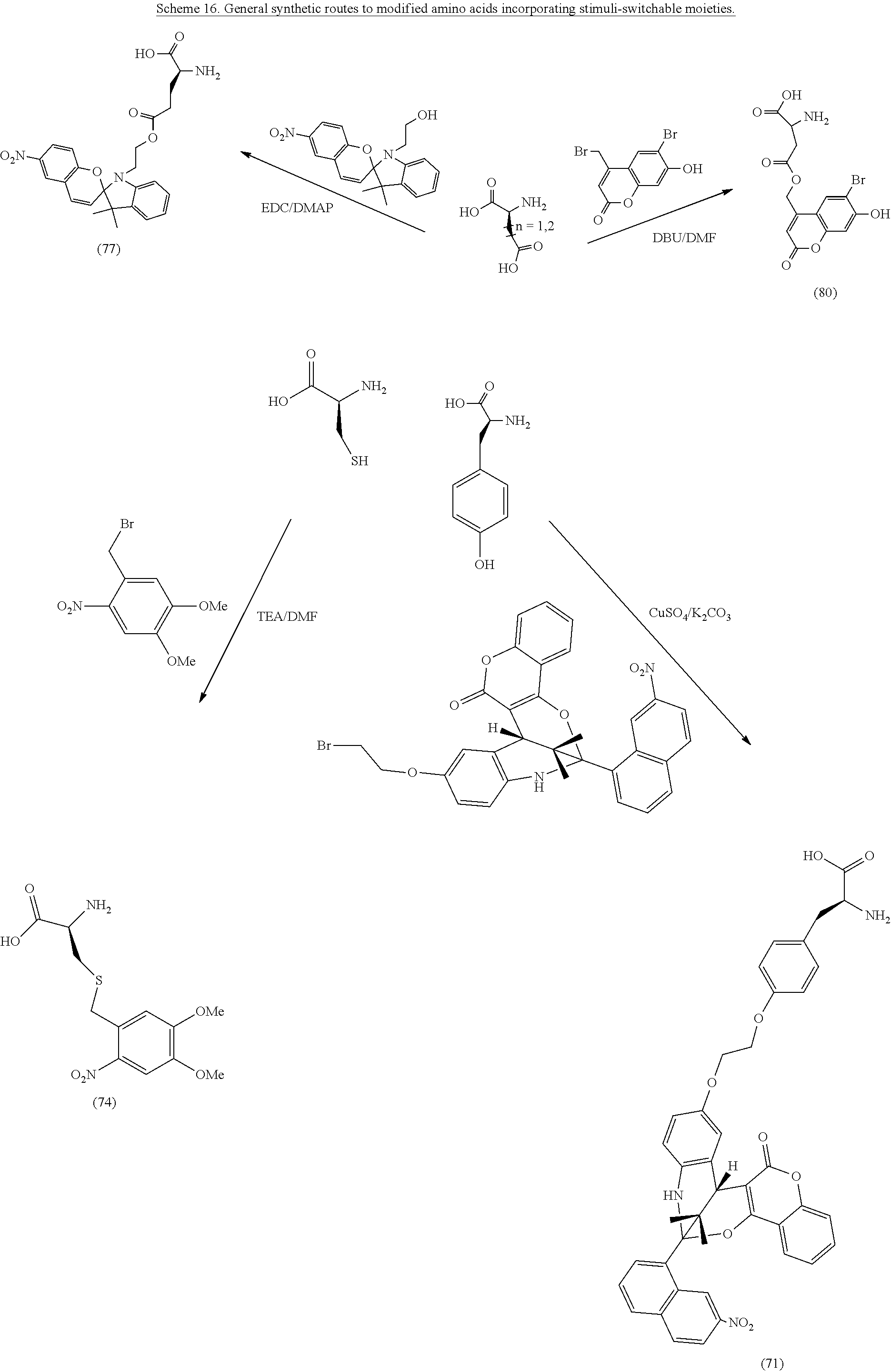

In some embodiments, the stimuli-switchable moiety that is coupled to the amino acid comprises a spiropyran, spirooxazine, coumarin, nitrobenzene, azobenzene, bisthienylethene, flugide, 2-diazo-1,2-napthoquinone-5-sulfonyl-methylacrylamide, napthacene derivative, or the like.

In some embodiments, the modified amino acids incorporating a stimuli-switchable moiety are used to directly synthesize peptides incorporating the stimuli-switchable moiety. The modified amino acids can then be used to synthesize peptides incorporating the stimuli-switchable moiety (or more than one stimuli-switchable moiety) at specific location(s) within the peptide sequence using standard peptide synthesis techniques, modified as necessary to avoid damaging or cleaving the stimuli-switchable moiety. For example, protected monomers (70), (73), (76) or (79) above could be used in conventional peptide synthesis. It would be within the expected ability of the person of ordinary skill in the art to synthesize peptides and use conditions that would not damage or cleave the stimuli-switchable moiety. In the case of a napthacene-based stimuli-switchable moiety, for example having the structure (21), (22) or (23), strong acidic and strong basic conditions would be avoided in peptide synthesis.

An example of an initiator that can be used for the solid-phase synthesis of peptides incorporating amino acids bearing stimuli-switchable moieties is, for example Rink amide resin or Wank resin, which are conventionally used for peptide synthesis.

In some embodiments, the peptide is synthesized directly on a solid surface on which the final stimuli-switchable polymer is to be provided for its ultimate end use. For example, in some embodiments, a silicon wafer could be functionalized so that the desired peptide could be synthesized directly on the silicon wafer, incorporating one or more amino acids bearing a stimuli-switchable moiety at desired location(s) within the peptide sequence. The silicon wafer bearing the stimuli-switchable polymer could then be used in any desired end application. Other solid surfaces on which a peptide bearing stimuli-switchable moieties might be directly synthesized from amino acids include silica nanoparticles, cadmium selenide, ferric oxide nanoparticles, zinc oxide, titanium dioxide, manganese oxide nanoparticles, carbon nanotube membranes, polycarbonate membranes, polyimide membranes, polyethylene terephthalate membranes, and the like. In some embodiments, the solid surface is functionalized for peptide synthesis by initially oxidizing the starting material (for example, by acid hydrolysis or plasma treatment). The resultant hydroxyl group is used to react with an FMOC protected amino-acid to initiate peptide synthesis. Thus, the functionalized solid surface can allow synthesis of a peptide incorporating amino acids having a desired sequence, including the incorporation of a modified amino acid bearing a stimuli-switchable moiety at any desired location within the peptide sequence, in the same manner as Rink amide resin or Wank resin. In some embodiments, the protected amino acids are not deprotected after peptide synthesis, as in some applications it may be desirable to retain the protecting groups on the peptide.

The use of modified amino acids incorporating a stimuli-switchable moiety to directly synthesize peptides allows for control of the peptide sequence, and for control of the location of the stimuli-switchable moiety (or multiple stimuli-switchable moieties) within the polypeptide.

Peptides incorporating stimuli-switchable moieties at specific locations have potential application, for example in the modulation of peptide-binding affinities. For example, some peptides could bind with a desired target when the stimuli-switchable moiety is in a first state and release the desired target when the stimuli-switchable moiety is in a second state. Thus, for example in the case of a peptide incorporating a stimuli-switchable moiety that reversibly switches from hydrophobic to hydrophilic upon exposure to light, the peptide could be made to selectively bind or release a target in response to the application of light having a suitable wavelength to cause the stimuli-switchable moiety to switch from hydrophilic to hydrophobic. Or, in the case of a peptide incorporating a stimuli-switchable moiety that irreversibly switches from hydrophobic to hydrophilic upon exposure to light, the peptide could bind a desired target, the bound peptide-target complex could be subjected to further processing (e.g. recovery from a solution), and then the target could be released from the peptide by the application of light having a suitable wavelength to cause the irreversible switch of the stimuli-switchable moiety from hydrophobic to hydrophilic.

In alternative embodiments, modified amino acids incorporating a stimuli-switchable moiety can be modified to be suitable for use as monomers for polymerization. For example, the modified amino acids incorporating a stimuli-switchable moiety can be converted to their corresponding N-carboxyanhydride. The N-carboxyanhydride monomers can be reacted together to form a polymer.

In some embodiments, photo-switchable amino acid N-carboxyanhydrides, suitable for preparation of polymers by surface-initiated polymerization are prepared by the reaction of photo switchable amino acids (which are aspartic acid, cysteine, glutamic acid, or tyrosine in some example embodiments) with triphosgene, to form the corresponding N-carboxyanhydrides.

Scheme 12 shows a reaction scheme for the production of Napthacene-Tyr-NCA (72), via a α-N-protected active amino ester. The modified Napthacene-Tyr (71) amino acid is reacted with triphosgene to arrive at the desired Napthacene-Tyr-NCA (72) N-carboxyanhydride monomer.

In another example embodiment, the N-carboxyanhydride of nitrobenzyl-modified cysteine (75) is obtained from nitrobenzyl-modified cysteine (74) by reaction with triphosgene according to Scheme 13.

In another example embodiment, the N-carboxyanhydride of spiropyran-modified glutamic acid (78) is obtained from spiropyran-modified glutamic acid (77) by reaction with triphosgene according to Scheme 14.

In another example embodiment, the N-carboxyanhydride of coumarin-modified aspartic acid (81) is obtained from coumarin-modified aspartic acid (80) by reaction with triphosgene according to Scheme 15.

The resultant N-carboxyanhydrides can be used with an appropriate initiator to synthesize polymers on a solid surface via ring opening polymerization. Any suitable initiator and scheme for carrying out ring opening polymerization, as would be within the knowledge of one skilled in the art, could be used. One example of an appropriate initiator is the structure shown as (1) in Scheme 8. In some embodiments, NCA polymerizations are initiated via the surface grafting, conformation, and orientation of various amino acids polymerized in solution from (γ-aminopropyl)triethoxysilane (APS) coated silicon wafers, for example as shown in Schemes 17, 18 and 19. NCA polymerizations can be initiated using many different nucleophiles and bases, the most common being primary amines and alkoxide anions. Primary amines, being more nucleophilic than basic, are good general initiators for polymerization of NCA monomers. Potential pathways of NCA polymerization are the so-called “amine” and the “activated monomer” (AM) mechanisms. The amine mechanism is a nucleophilic ring-opening chain growth process where the polymer could grow linearly with monomer conversion if side reactions were absent.

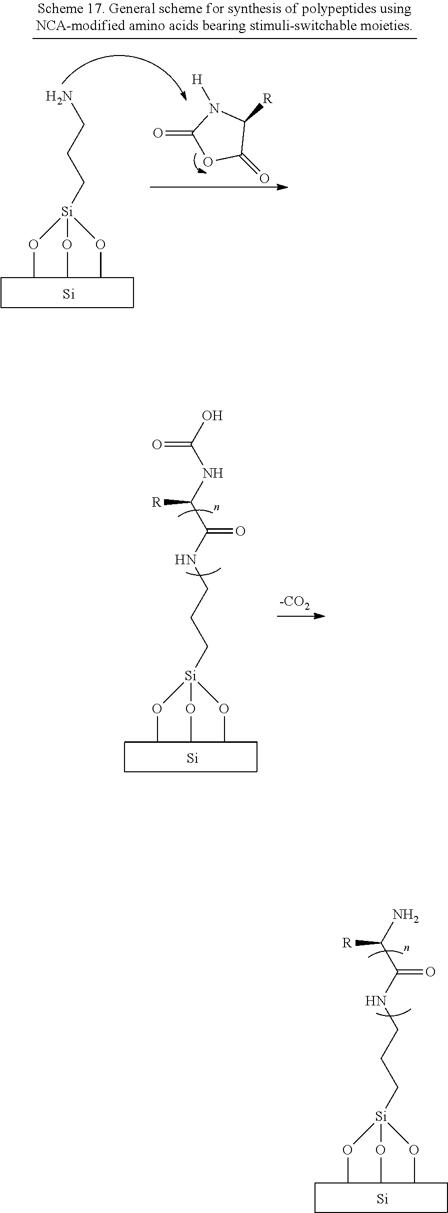

Schemes 18 and 19 show specific example embodiments illustrating the synthesis of a silicon surface bearing polyglutamate bearing a spiropyran switchable moiety, and a silicon surface bearing polytyrosine bearing a napthacene switchable moiety, respectively.

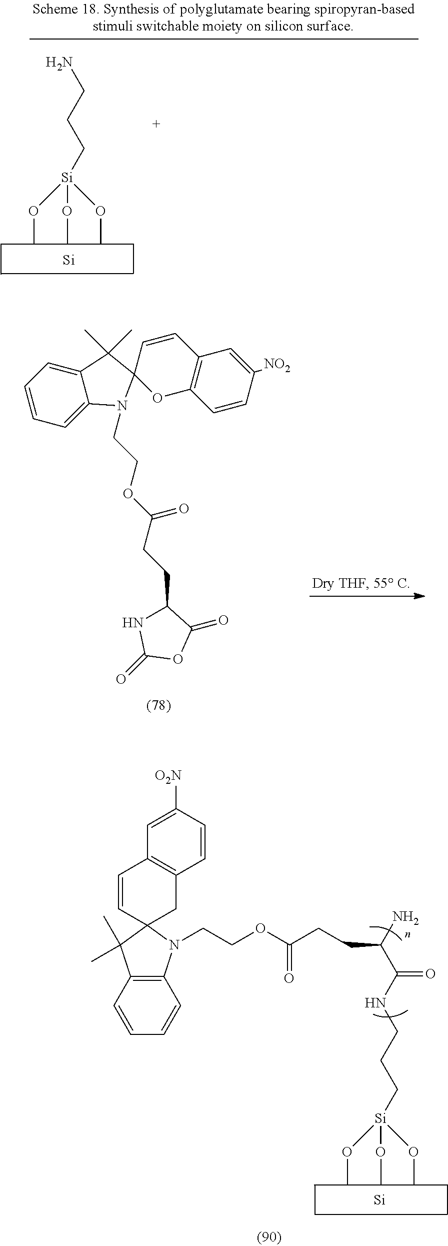

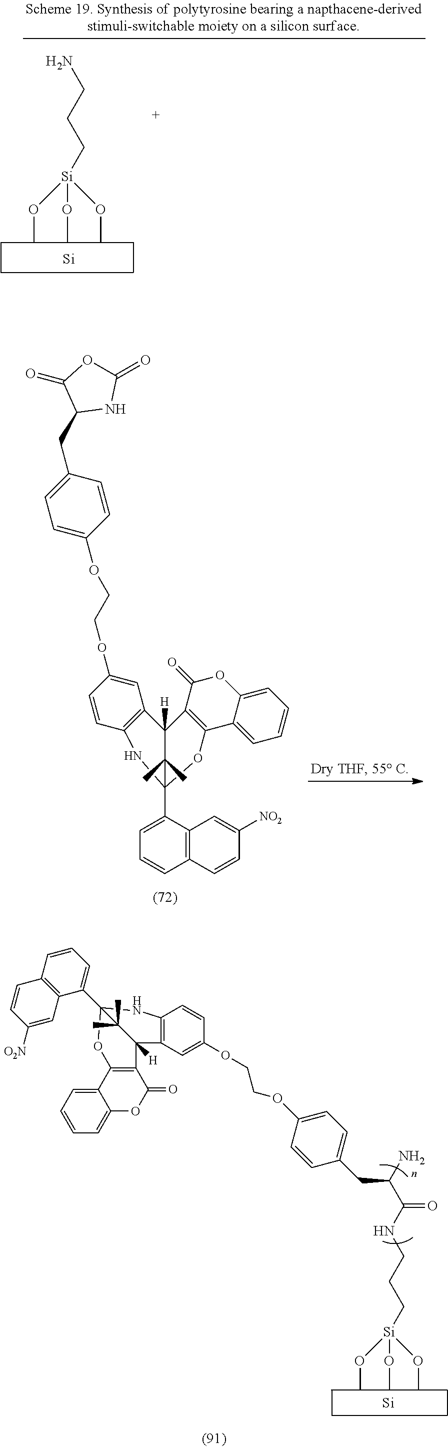

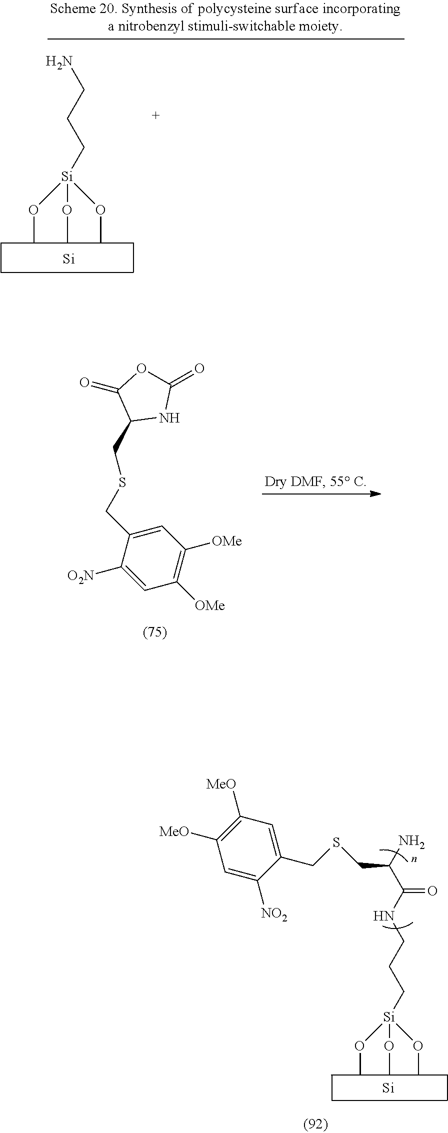

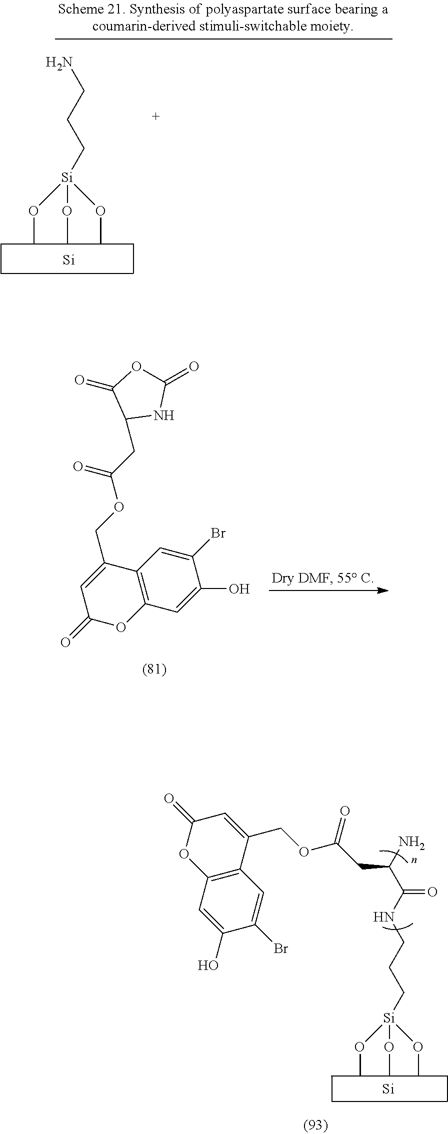

Certain embodiments of the invention are further described with reference to the following examples, which are intended to be illustrative and not limiting in nature.

### EXAMPLES

### Materials

2-bromopropionyl bromide (97%), p-anisidine (98%), 1,2-dichloroethane (98%), isobutyraldehyde (99%), 1-nitronaphthalene-3-carbaldehyde (95%), 4-Hydroxycoumarin (99%), p-Toluenesulfonic acid monohydrate (98.5%), CuBr (99.999%), CuCl (99.999%), N,N,N′,N″,N″-pentamethyldiethylenetriamine (PMDETA, 99%), propargyl bromide (99%) 4-dimethylaminopyridine (DMAP, 99%), and triethylamine (TEA, 99.5%) 2-nitrobenzaldehyde (95%), aminopropyltriethoxysilane (ATEPS, 99%), 2-bromo-isobutryl bromide (98%), 1-ethyl-3-(3-dimethylaminopropyl)carbodiimide (98%), and sodium azide (NaN₃) were purchased from Aldrich and used as received. Methacroyl chloride (Aldrich, 99%), and glycidyl methacrylate (GMA, Aldrich, 97%) were purified by passing through an alumina column and stored under N₂ at −15° C. for use. Solvents were distilled before use and other reagents were used without further purification.

### Instruments and Measurements

600 MHz ¹H-NMR spectra were recorded on a Bruker Fourier transform spectrometer, using D₂O and CDCl3 as solvent. Contact angles were performed on a Rame'-hart Model 400 Goniometer. The surface morphologies of photo-responsive polymer brushes were performed in the “Tapping mode” obtained using Atomic force microscopy (using Bruker Icon AFM Santa Barbara, Calif.) system. Electrically conducting SCM-PIT probe (Bruker, Santa Barbara, Calif.) with resonance frequency 64.1 kHz, spring constant of 2.8 N/m, tip of radius of curvature (a) of 20 nm.

### Example 1.0—Synthesis of Napthacene-Based Stimuli-Switchable Moiety

As shown in Scheme 4, an acrylic monomer incorporating a novel napthacene-based photoswitchable moiety (25) was prepared by multi step organic synthesis. At first, to a solution of 4-aminophenol (3.12 mmol) in methylene chloride (10 mL) was added propargyl bromide (3.14 mmol) and anhydrous K₂CO₃ (4.32 mmol). After the mixture was stirred at reflux for overnight, the solution was then filtered and concentrated. The resulting crude product was purified by column chromatography in methylene chloride/hexanes to give a yellow solid with a 90% yield. Then, the resulting product in methylene chloride was added nitrobenzaldehyde/nitro naphthaldehyde/and anhydrous magnesium sulfate. After the mixture was stirred at room temperature for one hour, the solution was then filtered and concentrated and was crystallized in methylene chloride/hexanes to give a yellow solid. In the next step, the compound (31) in THF (5 mL) was added Yb(OTf)₃ (0.09 mmol) and isobutyraldehyde (0.74 mmol). The resulting solution was stirred at room temperature for overnight, water (10 mL) was then added to the mixture and the product was extracted with methylene chloride. The combined organic extracts were dried over MgSO₄, filtered, and concentrated and purified by column chromatography (1:5 EA/hexanes) to give a brown solid. Finally, to a solution of (32) (2.4 mmol) in 1,2-dichloroethane (20 mL) was added 4-hydroxycoumarin (2.4 mmol) and a catalytic amount of p-TsOH. After the mixture was refluxed for overnight, water (15 mL) was added to quench the reaction and the product was extracted with methylene chloride. The combined organic extracts were dried over MgSO₄, filtered and concentrated and was purified by column chromatography (1:10 EA/hexanes) to give a yellowish solid.

### Example 1.1—Characterization of Napthacene-Based Stimuli-Switchable Moiety

The new photoswitchable moieties were characterized by ¹H NMR, with the following results.

### Naphthacene propargyl (25)

¹H NMR: δ(CDCl₃) 9.19, 9.03, 8.38-8.3315 (m, 2H), 7.26 (m, 2H), 7.1 7.0˜6.62 (m, 2H,), 6.51 (s, 1H), 6.31 (s, 1H), 5.22 (d, 2H), 4.8 (d, 2H), 4.5 (m, 2H O—CH₂), 2.5 (m, Propargyl), 2.0 (s, 1H), 1.7 (s, 2H), 1.2 (S, 1H)

### Naphthacene methacrylate (24)

¹H NMR: δ(CDCl₃) 9.19 (S, 1H), 8.38 (d, 2H), 8.31 (d, 2H), 7.86 (m, 2H), 7.66 (d, 1H) 7.29˜7.19 (m, 2H,), 6.67 (s, 1H), 6.221 (s, 1H), 4.69 (m, 2H, —O—CH2), 4.4 (m, 2H, double bond), 4.12 (m, 2H, double bond), 3.6 (s, 1H), 3.24 (s, 1H), 2.08 (s, 3H, —CH3), 1.15 (s, 6H, —CH3)

### Naphthacene glutamate (28)

¹H NMR: δ(CDCl₃) 9.19 (S, 1H), 8.38 (d, 2H), 8.13 (d, 2H), 8.31 (d, 2H), 7.86 (m, 2H), 7.66 (d, 1H) 7.29˜7.19 (m, 2H,), 6.78 (d, 1H), 6.78 (d, 1H), 3.61 (s, 2H), 3.37˜3.27 (m, 4H), 2.89 (m, 2H), 2.53 (m, 2H), 1.53 (s, 6H).

### Naphthacene azide (27)

¹H NMR: δ(CDCl₃) 9.19 (S, 1H), 8.38 (d, 2H), 8.13 (d, 2H), 8.31 (d, 2H), 7.86 (m, 2H), 7.66 (d, 1H) 7.20˜7.14 (m, 2H,), 6.65 (m, 2H), 4.01 (m, 2H), 3.38 (m, 2H), 1.15 (s, 6H).

### Example 1.2—Synthesis of Surface-Bound Polymer Incorporating Napthacene-Based Stimuli-Switchable Moiety

A polymer incorporating a napthacene-based stimuli-switchable moiety having the structure (22) was prepared on a silicon surface using a post-polymerization modification approach following the reaction set forth in Scheme 6 using ATRP. Briefly, a post-modification strategy was used in which poly(azide reminated hydroxyl acrylate) brushes were generated via surface-initiated controlled radical polymerization and sequentially functionalized with the switchable moieties using a click reaction. Surface-initiated Atom Transfer Radical Polymerization (SI-ATRP) initiated on a bromo-isobutryl initiator attached on silicon wafer. At first, for synthesizing an amine terminated monolayer on silicon wafers, freshly diced (1×1 cm²) silicon wafers were used as substrates and were rinsed with Milli-Q water and ultrasonically cleaned with subsequently warm (30° C.) ethanol and dichloromethane (DCM). After these pre-cleaning steps, the silicon wafers were treated with Piranha solution (H₂O₂/H₂SO₄, 3:7) for 30 minutes, followed by thoroughly washing with a large quantity of Milli-Q water, again ultrasonically cleaned with methanol and methanol/toluene and toluene for 5 minutes. Then they were immediately silanized with freshly distilled (γ-aminopropyl)triethoxy silane (2%) in dry toluene. Then, the silicon wafers were cleaned with toluene, MeOH and finally thoroughly dried under vacuum. The slides were immersed in a 100 mL flask containing 3 mL of triethylamine (TEA) in 70 mL of dichloromethane (DCM) at 0° C. Then 2.5 ml of bromoisobutyryl bromide and 10 mL of DCM were combined and added in drop wise. The reaction was continued for 2 hrs at 0° C. and room temperature for overnight. Finally the initiator grafted silicon wafers were cleaned with DCM and dried.

For polymerization, a solution containing 140 ml of dry acetone and glycidylacrylate (monomer) was bubbled with argon to remove oxygen for 30 minutes. 60 mg of copper (I) bromide, 120 mg of N,N,N′,N″,N″-pentamethyldiethylenetriamine (PMDETA) were then added to the solution. This ATRP (as described in U.S. Pat. Nos. 5,763,548 and 5,789,487, which are incorporated by reference herein) solution was then sonicated for a period of 10 minutes. The initiator grafted silicon wafers were then immersed into this solution and allowed to react at 50° C. for 24 hours. The wafers were then removed from the solution and washed thoroughly with THF and MeOH and dried. The poly(glycidyl acrylate) was efficiently opened with sodium azide in the presence of ammonium chloride in DMF at 50° C. to prepare a polymer brush suitable for grafting the stimuli-switchable propargyl naphthacene monomers using click linking chemistry. This click-type reaction led to the formation of a polymer brush with distributed units of the corresponding 1-hydroxy-2-azido functional group in high yields. These azide-containing polymer brushes were further functionalized in a second click reaction conducted at room temp. CuBr/N,N,N′,N″,N″-pentamethyldiethylenetriamine-catalyzed 1,3-dipolar cyclo-addition of propargyl naphthacene yielded grafted polymeric brushes with photo-responsive side chains bearing a napthacene-based stimuli-switchable moiety.

A polymer incorporating a napthacene-based stimuli-switchable moiety having the structure (22) was prepared on a silicon surface using a pre-polymerization modification approach following the reaction set forth in Scheme 7 using ATRP. Briefly, a pre-polymerization modification strategy was used in which Surface-initiated Atom Transfer Radical Polymerization (SI-ATRP) was initiated on a bromo-isobutryl initiator attached on a silicon wafer. At first, for synthesizing of an amine terminated monolayer on silicon wafers, freshly diced (1×1 cm²) silicon wafers used as substrates and were rinsed with Milli-Q water and ultrasonically cleaned with subsequently warm (30° C.) ethanol and dichloromethane (DCM.). After these pre-cleaning steps, the silicon wafers were treated with Piranha solution (H₂O₂/H₂SO₄, 3:7) for 30 minutes, followed by thoroughly washing with a large quantity of Milli-Q water. The wafers were again ultrasonically cleaned with methanol and methanol/toluene and toluene for 5 minutes. Then they were immediately silanized with freshly distilled (γ aminopropyl)triethoxy silane (2%) in dry toluene. Then, silicon wafers were cleaned with toluene, MeOH and finally thoroughly dried under vacuum. The slides were immersed in a 100 mL flask containing 3 mL of triethylamine (TEA) in 70 mL of dichloromethane (DCM) at 0° C. Then 2.5 ml of bromoisobutyryl bromide and 10 ml of DCM were combined and added in drop wise. The reaction was continued for 2 hours at 0° C. and room temperature for overnight. Finally the initiator grafted silicon wafers were cleaned with DCM and dried. For polymerization, a solution containing 140 ml of dry THF and naphthacene methacrylate monomer was bubbled with argon to remove oxygen for 30 minutes. 60 mg of copper (I) bromide, 120 mg of N,N,N′,N″,N″-Pentamethyldiethylenetriamine (PMDETA) were then added to the solution. This ATRP (as described in U.S. Pat. Nos. 5,763,548 and 5,789,487) solution was then sonicated for a period of 10 minutes. The initiator grafted silicon wafers were then immersed into this solution and allowed to react at 65° C. for 24 hours. The wafers were then removed from the solution and washed thoroughly with THF and MeOH and dried to yield the final surface-bound polymer incorporating a napthacene-based stimuli switchable moiety.

### Example 1.3—Hydrophobic-Hydrophilic Behavior of Napthacene-Based Stimuli-Switchable Moiety

As shown in FIG. 2, in one example contact angle study using a polymeric surface prepared from a napthacene-based stimuli-switchable moiety, napthacene methacrylate, prepared using the pre-polymerized ATRP strategy shown in Scheme 7, the contact angle changed from approximately 90° when the napthacene-based stimuli-switchable moiety was in the hydrophobic state to approximately 27° when the napthacene-based stimuli-switchable moiety was in the hydrophilic state. The application of ultraviolet light having a wavelength of approximately 365 nm was used to switch the napthacene-based stimuli-switchable monomer from its hydrophobic configuration (left panel) to its hydrophilic configuration (right panel).

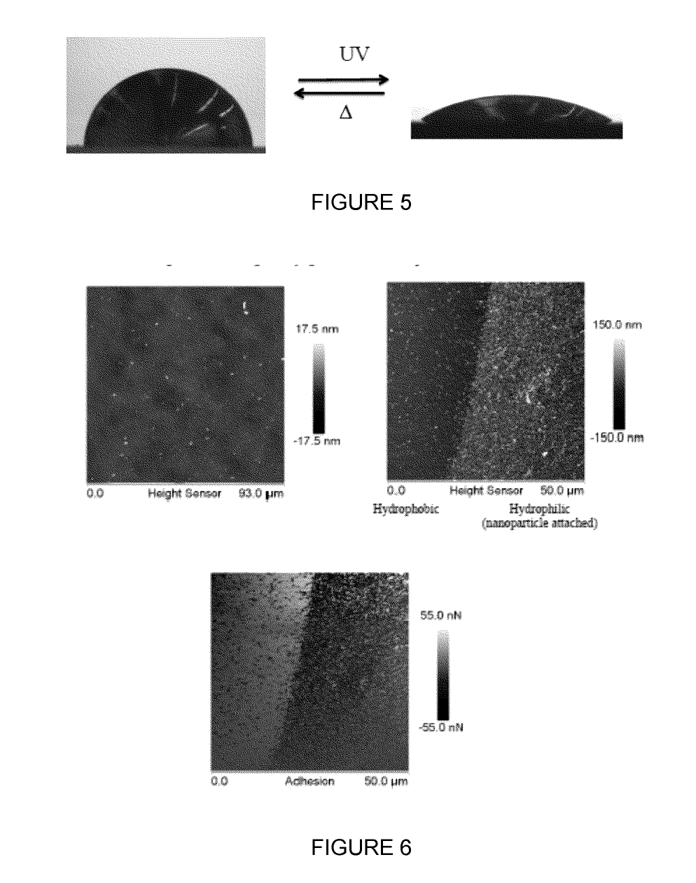

FIG. 5 shows a further experiment demonstrating the degree of change in hydrophobicity achieved by the napthacene-based stimuli-switchable moiety having the structure (22). The polymeric surface tested in FIG. 5 was prepared using polymerization of napthacene acrylate monomers according to Scheme 7, as described in Example 1.2. Comparative polymeric surfaces incorporating spiropyran-based, nitrobenzene-based and coumarin-based photoswitchable moieties were prepared in a similar fashion by polymerization of their acrylate monomers. Results of the contact angle measured for each polymeric surface in its hydrophobic state (no irradiation) and hydrophilic state (with irradiation) are shown in Table 3, for both the advancing (maximum) contact angle θ_{adv} and the receding (minimum) contact angle θ_{rec}.

TABLE 3
Comparative contact angles for stimuli-switchable polymeric surfaces.

| Contact | Napthacene | | Spiropyran | | Nitrobenzene | | Coumarin | |
|---|---|---|---|---|---|---|---|---|
| Angle | No irr. | With irr. | No irr. | With irr. | No irr. | With irr. | No irr. | With irr. |
| θ_{adv} | 109.8 ± 1.2 | 20.8 ± 1.2 | 93.8 ± 1.2 | 18.8 ± 1.2 | 76.0 ± 1.2 | 15.8 ± 1.2 | 81.8 ± 1.2 | 12.8 ± .12 |
| θ_{rec} | 89.5 ± 1.5 | 15.4 ± 1.7 | 72.2 ± 1.8 | 17.5 ± 0.6 | 68.5 ± 1.3 | 11.5 ± 0.4 | 72.2 ± 1.1 | 10.2 ± 0.8 |

As can be seen from Table 3, the polymeric surface incorporating a napthacene-based stimuli-switchable moiety is considerably more hydrophobic than surfaces incorporating typical stimuli-switchable moieties based on spiropyran, nitrobenzene, and coumarin, and also exhibits a large magnitude of change in contact angle when switched from the hydrophobic to the hydrophilic state (e.g. approximately 1.5 times the magnitude of change in contact angle for the polymer bearing a nitrobenzene stimuli-switchable moiety, as determined based on the change in θ_{adv}. The switching of nitrobenzene and coumarin from the hydrophobic to the hydrophilic state is not reversible, while the switching of the napthacene-based stimuli-switchable moiety is reversible. The results of this experiment show that napthacene-based stimuli-switchable moieties and surfaces incorporating same are reversibly switchable with a very large change in hydrophobicity upon changing between the hydrophobic and hydrophilic states. Polymeric surfaces incorporating such a stimuli-switchable moiety have potential application in fields such as micropatterning, selective adhesion, microfluidics, and the like, which require a large change in hydrophobicity between the hydrophilic and hydrophobic states of the stimuli-switchable moiety. In some embodiments, using heat to switch the stimuli-switchable moieties to the hydrophobic configuration can allow for fast switching. In some embodiments, for example during micropatterning, light can be used to locally heat the surface to cause the stimuli-switchable moieties to switch back to the hydrophobic state.

Based on the results described above for the napthacene-based stimuli-switchable moiety having the structure (22), it can be soundly predicted that the other napthacene-based stimuli-switchable moieties having the structure (21), (22) or (23) can be used as stimuli-switchable moieties. In particular, the basic structure for stimuli-induced hydrophilic-hydrophobic change is the same for all three structures. The reversible stimuli-induced hydrophilic change is attributed to an equilibrium between a closed and colorless (nitro-naphacene or nitrobenzene or nitro-pyrlene) hydrophobic form and an open, colored hydrophilic form. The colored, hydrophilic form is formed due to the highly localized electron distribution in the zwitterion biradical, which is an equilibrium mixture of geometrical conformations. These structures can also undergo light-mediated indazole ring formation to return to the neutral, hydrophobic state.

The contact angle studies described above examining the manipulation of surface energy by light to influence the water wettability of stimuli-responsive polymer surfaces. These exemplary studies were conducted on stimuli-responsive polymers grafted on a silicon wafer surface, which has much smoother surface than glass, metal, or the like. However to achieve higher hydrophobic properties (such as super hydrophobicity), other surfaces can combine some level of inherent hydrophobicity with surface geometry; for example, there can be significant differences in the behavior and functionality of rectilinear and curved features. Thus, in some embodiments, modifying the properties of the surface, for example by increasing surface roughness, can potentially enhance the hydrophobic properties of a napthacene-based stimuli-switchable moiety having the structure (21), (22) or (23).

### Example 2.0—Lactone-Based Monomers Incorporating Stimuli-Switchable Moieties

All chemicals were purchased from Sigma Aldrich, TCI America and VWR used as received. Anhydrous solvents were used all the reactions. All silane-coupling reagents were purified by vacuum distillation. All reactions were stirred magnetically, under an argon atmosphere, unless otherwise noted, and monitored with analytical TLC (Merck Kieselgel 60 F254). Column chromatography was carried out with silica gel 60 particle size 0.063-0.210 mm. NMR spectra were measured in Bruker 600 MHz. Chemical shifts were reported in the δ scale relative to tetramethylsilane (TMS) as 0.00 ppm for 1 H (CDCl₃) and residual CHCl₃ (7.26 ppm for ¹H), as internal reference.

In general, photo-switchable lactones are made by a multi-step organic synthesis. At first, a lactone bearing a carboxylic acid moiety was synthesized. 1.1 g of Wilkinson's catalyst (Rh/C) was added to a solution of 11.94 g of ethyl p-hydroxycinnamate in ethyl acetate (100 mL) and the mixture was stirred under atmospheric pressure of H₂ for five days at room temperature. Then, the reaction mixture was filtered through a Celite pad eluting with ethyl acetate, and concentrated under reduced pressure. The residue was purified by silica gel column chromatography (EA/Hex 1:3) to afford product as colorless oil. Then 48 mg of LiAlH₄ was added to a stirred solution of above product in THF at 0° C. and the mixture were stirred for 4 hours at room temperature. Then, 0.15 mL of H₂O and 15% aqueous NaOH was added in to the reaction mixture cooled at 0° C., and the reaction was continued for 10 minutes. An additional 0.5 mL of H₂O was dropped into the reaction mixture and further stirring was continued for 30 minutes at 0° C. After MgSO₄ was added, the reaction mixture was filtered through a Celite pad and concentrated under reduced pressure. The product was purified by silica gel column chromatography (EA/Hex 1:1) to afford diol (800 mg, 96%) as a colorless solid.

### Example 2.1—Lactone-Based Monomers Incorporating Napthacene-Based Stimuli-Switchable Moiety

Lactone-based monomers bearing the new napthacene-based stimuli-switchable moiety described in this specification was synthesized by multi-step organic synthesis starting from a lactone bearing a carboxylic acid functional group at the desired point of incorporation of the stimuli-switchable moiety (prepared in accordance with the method described in U.S. Pat. No. 8,470,958, which is incorporated by reference herein) according to the general reaction of Scheme 10. In a 100 mL, two-necked round bottom flask, 0.02 mmol of lactone carboxylic acid and the selected photo-switchable napthacene moiety bearing an alcohol functional group as X (synthesized using DMAP EDC chemistry), 0.228 mmol of N-(3-Dimethylaminopropyl)-N′-ethylcarbodiimide hydrochloride (EDC), 0.02 mmol of 4-dimethylaminopyridine (DMAP) in 70 mL of dichloromethane (DCM) at 0° C. for two hours and stirred at room temperature for overnight. The reaction flask was protected from light by wrapping with aluminum foil during the reaction. The salt formed during the reaction was filtered, solvent was removed and organic solution was extracted with water (3×20 mL) and dried over MgSO₄. Finally purification was done by column chromatography using eluent (EA/Hex 1:6-3:2).

¹H NMR for (52) napthacene-based moiety lactone: δ(CDCl₃) 9.09 (s, 1H), 8.39-8.33 (m, 2H), 8.13 (d, 1H), 8.03 (d, 2H), 7.28 (d, 1H), 7.14 (d, 2H), 6.7˜6.62 (m, 2H,), 4.70 (m, 2H), 4.71 (m, 1H), 4.28 (m, 2H, lactone O—CH₂), 4.13 (m, 2H), 3.75 (m, 2H O—CH₂), 3.60 (s, 1H), 2.45˜2.26 (m, lactone), 1.80˜1.76 (m, 6H), 1.7 (m, 4H), 1.4 (S, 6H, —CH3).

### nitro-pyerlene lactone monomer (60)

¹H NMR: δ(CDCl₃) 9.03 (s, 1H), 8.39-8.15 (m, 2H), 8.01 (d, 1H), 7.81 (m, 1H), 7.29- (m, 2H), 6.7˜6.4 (m, 2H,), 4.72 (m, 2H), 4.61 (m, 1H), 4.28 (m, 2H, lactone O—CH₂), 4.17 (m, 2H), 3.75 (m, 2H O—CH₂), 3.60 (s, 1H), 2.45˜2.27 (m, lactone), 1.81˜1.52 (m, 6H), 1.7 (m, 4H), 1.2 (S, 6H, —CH3).

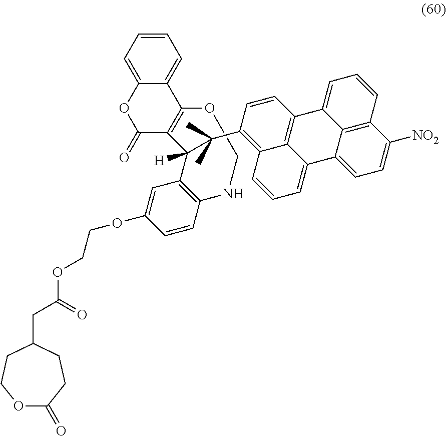

### nitrobenzyl lactone (51)

¹H NMR: δ(Acetone) 7.45 (s, 1H), 7.05 (S, 1H), 4.49 (t, 2H, O—CH₂) 4.42˜4.36 (m, 2H, —O—CH₂), 3.91 (S, 6H, O—CH₃), 3.8 (d, 2H), 3.7˜6.4 (m, 2H,), 3.47˜3.40 (m, 2H), 3.01˜2.94 (m, 2H), 2.46˜2.37 (m, 2H, lactone O—CH2), 1.81˜1.52 (m, 6H), 1.6 (m, 2H).

### Coumarin lactone (53)

¹H NMR: δ(CDCl₃) 7.65 (s, 1H), 6.79 (S, 1H), 6.14 (s, 1H, coumarin), 6.18 (s, 1H, coumarin), 4.56˜4.5 (m, 2H, O—CH₂), 4.32 (d, 2H), 3.87˜3.71 (m, 4H, —CH₂), 3.30 (m, 2H, —CH₂), 3.05 (m, 2H), 2.94 (m, 2H, O—CH₂ aromatic), 2.44˜2.33 (m, 2H, lactone O—CH₂), 2.15 (m, 2H), 1.95˜1.88 (m, 2H), 1.71 (m, 2H), 1.44˜1.49 (m, 2H, lactone).

### Spiropyran lactone (54)

¹H NMR: δ(CDCl₃) 8.42 (s, 1H), 8.09 (S, 1H), 7.04 (d, 1H), 6.76 (d, 2H), 6.64 (d, 1H), 6.51 (d, 2H), 6.30 (s, 1H), 5.94 (d, 1H), 4.52 (m, —CH₂), 4.32 (m, 2H, O—CH₂), 3.88˜3.57 (m, 6H), 3.22 (m, 2H, —CH₂), 3.00 (m, 2H, N—CH₂), 2.44˜2.35 (m, 2H, —CH₂), 2.2 (m, 2H, O—CH₂), 1.934˜1.78 (m, 6H, lactone O—CH₂), 1.36 (s, 6H).

### Example 2.2—Synthesis of Polymers Incorporating Stimuli-Switchable Moieties Using Lactone-Based Monomers

Polymerization was carried out according to the method described in Scheme 11 to confirm that the synthesized monomers described in Example 2.1 could be used to form a polymer incorporating a stimuli-switchable moiety. Polymerization efficiency observed was generally above 80%.

### Example 3.0—Preparation of Modified Amino Acids Incorporating Stimuli-Switchable Moieties

Modified amino acids having the structures shown below were synthesized, including spiropyran modified glutamic acid having structure (77), nitrobenzyl modified aspartic acid having the structure (82), coumarin modified aspartic acid having the structure (80), nitrobenzyl modified cysteine having the structure (74), and napthacene modified tyrosine having the structure (70).

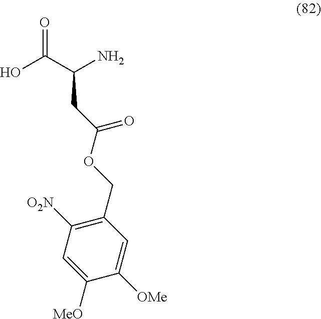 [SMILES: COc1cc(COC(=O)C[C@H](N)C(=O)O)c([N+](=O)[O-])cc1OC]

### Example 3.1—Synthesis and Characterization of Spiropyran L-Glutamic Acid (77)

Spiropyran-L-glutamic acid (77) was synthesized in a three step organic synthesis according to Scheme 14. In a 250 mL three necked round bottom flask charged with Boc-L-glutamic acid 1-t butyl ester (68 mmol), hydroxyl ethyl spiropyran (68 mmol), EDC (71 mmol), and DMAP (68 mmol) in 150 mL dichoromethane were stirred under argon at 0° C. for two hours and stirred at room temperature for overnight. The reaction flask was protected from light by wrapping aluminum foil during the reaction. The salt formed during reaction was filtered, solvent was removed and organic solution was extracted with water (3×70 mL) and dried over MgSO₄. Finally purification was done by column chromatography using eluent (EA/Hex 1:5). Then, the resulting product (76) in methylene chloride was deprotected by added trifluro acetic acid (TFA). After stirring at room temperature until starting material was consumed (TLC monitoring), the solution was concentrated in vacuo. When the uncharged, neutralized amino acid (77) was the desired product, the solution was extracted with water, saturated aqueous NaHCO₃ solution, water, and brine. The combined organic layers were dried over MgSO₄, filtered, and evaporated in vacuo to yield the crude product in quantitative yield.

¹H NMR (CDCl3) δ=8.4 (s, 1H), 8.15 (s, 1H), 7.2 (m, 1H), 6.8 (m, 1H), 6.5 (m, 2H), 5.9 (d, 2H), 5.09 (m, 1H), 4.01 (m, 2H, CH₂), 3.59 (m, 2H, CH₂), 1.9 (broad, 1H, NH₂), 1.4 (s, 6H, CH₃).

Nitrobenzyl-modified glutamic acid having the structure (83)

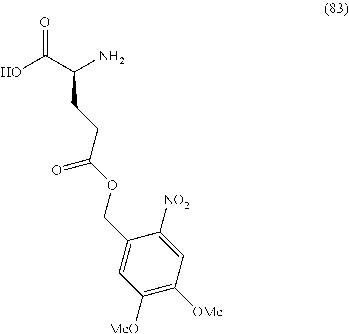 [SMILES: COc1cc(COC(=O)CC[C@H](N)C(=O)O)c([N+](=O)[O-])cc1OC]

was obtained in a similar fashion. Briefly, in a 100 mL three necked round bottom flask charged with Boc-L-glutamic acid 1-t butyl ester (62 mmol), 4,5-Dimethoxy-2-nitrobenzyl bromide (62 mmol), EDC (66 mmol), and DMAP (61 mmol) in 50 mL dichoromethane was stirred under argon at 0° C. for two hours and stirred at room temperature for overnight. The reaction flask was protected from light by wrapping with aluminum foil during the reaction. The salt formed during reaction was filtered, solvent was removed and organic solution was extracted with water (3×30 mL) and dried over MgSO₄. Finally purification was done by column chromatography using eluent (EA/Hex 1:3). Then, the resulting product in methylene chloride was deprotected by added trifluro acetic acid (TFA). After stirring at room temperature until starting material was consumed (TLC monitoring), the solution was concentrated in vacuo. When the uncharged, neutralized amino acid was the desired product, the solution was extracted with water, saturated aqueous NaHCO₃ solution, water, and brine. The combined organic layers were dried over MgSO₄, filtered, and evaporated in vacuo to yield the crude product in quantitative yield.

¹H NMR (CDCl3) δ=7.5 (s, 1H), 7.32 (s, 1H), 6.37 (m, 1H), 4.87 (d, 2H), 3.9 (s, 6H), 3.42 (m, 2H), 2.69 (broad, 2H, NH₂), 2.54 (m, 2H, —CH₂), 2.13 (m, 2H, —CH₂).

### Example 3.2—Synthesis and Characterization of N-(6-bromo-7-hydroxycoumarin-4-ylmethoxycarbonyl)-L-aspartic acid (80)

Bromo coumarin aspartic acid (80) was synthesized three step organic synthesis according to Scheme 15. In a 250 mL three necked round bottom flask charged with Boc-L-aspartic acid 1-t butyl ester (72 mmol), 6-bromo-7-hydroxycoumarin (72 mmol), EDC (76 mmol), DMAP (71 mmol) in 130 mL dichoromethane under argon at 0° C. for two hours and stirred at room temperature for overnight. The reaction flask was protected from light by wrapping with aluminum foil during the reaction. The salt formed during reaction was filtered, solvent was removed and organic solution was extracted with water (3×70 mL) and dried over MgSO₄. Finally purification was done by column chromatography using eluent (EA/Hex 1:2). Then, the resulting product in methylene chloride was deprotected by added trifluro acetic acid (TFA). After stirring at room temperature until starting material was consumed (TLC monitoring), the solution was concentrated in vacuo. When the uncharged, neutralized amino acid was the desired product, the solution was extracted with water, saturated aqueous NaHCO₃ solution, water, and brine. The combined organic layers were dried over MgSO₄, filtered, and evaporated in vacuo to yield the crude product in quantitative yield.

¹H NMR (CDCl3) δ=7.65 (s, 1H), 6.79 (S, 1H), 6.14 (s, 1H, coumarin), 6.18 (s, 1H, coumarin), 5.18 (m, 2H, O—CH2), 3.7 (s, 6H), 2.8 (d, 2H), 2.0 (broad, 1H, NH₂).

### Example 3.3—Synthesis and Characterization of Di methoxy 2-nitrobenzyl cysteine (74)

Di-methoxy 2-nitrobenzyl cysteine (74) was synthesized three step organic synthesis according to Scheme 13. In a 250 mL three necked round bottom flask charged with Boc-cysteine 1-t butyl ester (72 mmol), 4,5-Dimethoxy-2-nitrobenzyl bromide (72 mmol), EDC (76 mmol), and DMAP (71 mmol) in 130 mL dichoromethane were added and stirred under argon at 0° C. for two hours and stirred at room temperature for overnight. The reaction flask was protected from light by wrapping with aluminum foil during the reaction. The salt formed during reaction was filtered, solvent was removed and organic solution was extracted with water (3×70 mL) and dried over MgSO₄. Finally purification was done by column chromatography using eluent (EA/Hex 1:2). Then, the resulting product (73) in methylene chloride was deprotected by added trifluro acetic acid (TFA) to yield (74). After stirring at room temperature until starting material was consumed (TLC monitoring), the solution was concentrated in vacuo. When the uncharged, neutralized amino acid (74) was the desired product, the solution was extracted with water, saturated aqueous NaHCO₃ solution, water, and brine. The combined organic layers were dried over MgSO₄, filtered, and evaporated in vacuo to yield the crude product in quantitative yield.

¹H NMR (CDCl3) δ=7.54 (s, 1H), 7.05 (s, 1H), 7.2 (m, 1H), 4.7 (d, 2H), 3.7 (s, 6H), 3.4 (d, 2H), 2.0 (broad, 1H, NH₂).

### Example 3.4—Synthesis and Characterization of napthacene tyrosine (71)

Synthesis of tyrosine, bearing a napthacene-based stimuli-switchable moiety having the structure (22), (71) was carried out by multistep organic synthesis according to Scheme 12. In a 50 mL three necked round bottom flask charged with Fmoc-D-Tyr(tBu)-OH (100 mmol), the new photochromic naphthascene bromide (101 mmol) (having the general structure (22) wherein X is Br) was combined with K₂CO₃ (180 mmol) in 60 mL dry DMF and incubated under argon at 60° C. for overnight. The reaction flask was protected from light by wrapping with aluminum foil during the reaction. The DMF solvent was removed by vacuo and redissolved in DCM, filtered and organic solution was extracted with water (3×70 mL), dried over MgSO₄. Finally purification was done by column chromatography using eluent (EA/Hex 1:2). Then, the resulting product in methylene chloride was deprotected by added trifluroacetic acid (TFA) to yield (71). After stirring at room temperature until starting material was consumed (TLC monitoring), the solution was concentrated in vacuo.

¹H NMR (CDCl3) δ=9.09 (s, 1H), 8.39-8.33 (m, 2H), 8.13 (d, 1H), 8.03 (d, 2H), 7.29-7.19 (m, 2H), 6.92 (m, 2H,), 6.71 (m, 1H), 4.45 (m, 2H), 3.85 (m, 1H), 3.5 (m, 2H, O—CH2), 3.13 (m, 2H), 1.92 (d, 2H), 1.34 (S, 6H, —CH3).

### Example 4.0—Preparation of NCA-Amino Acids Incorporating Stimuli-Switchable Moieties

Generally, modified amino acids incorporating a stimuli-switchable moiety can be converted to their corresponding N-carboxyanhydride via reaction with triphosgene.

An N-carboxyanhydride of tyrosine bearing a napthacene-based stimuli switchable moiety as described in Example 1 (napthacene-Tyr-NCA (72)) was prepared according to Scheme 12. Triphosgene (2.65 g), Naphthasene-Tyr (71) (5.00 g), and tetrahydrofuran (THF) (60 mL) were charged to a 100 mL 3-neck round bottom flask fitted with a nitrogen purge, a magnetic stirrer, a condenser and a hot oil bath. The reaction mixture was heated to 60-65° C. until clear. The reaction mixture was continued for 6 hours and then cooled to ambient temperature, and nitrogen was bubbled through it to remove any excess phosgene. The solvent was then removed in vacuo. The crude product was crystallized from THF/hexane (2:5). The resulting product (72) was isolated by filtration and dried overnight in vacuo in dark.

An N-carboxyanhydride of aspartic acid bearing a coumarin-based stimuli switchable moiety (81) was prepared according to Scheme 15. The N-carboxyanhydride of 6-bromo-7-hydroxycoumarin-yl-methoxycarbonyl)-L-aspartate was synthesized single step synthesis from the corresponding amino acid (80). In a 100 mL three necked round bottom flask charged with magnetic stirrer bar, 6-bromo-7-hydroxycoumarin-4-ylmethoxycarbonyl)-L-aspartate (80) in 75 mL dry ethyl acetate under N₂ was added. The suspension was heated to reflux and an excess amount of triphosegene was added and the reaction was continued for 4-5 hours under N₂. The reaction flask was protected from light by wrapping aluminum foil during the reaction. The reaction solution concentrated and was then cooled to 5° C. to form the final product (81) as yellowish crystals.

¹H NMR (CDCl3) δ=8.06 (s, 1H), 7.4 (S, 1H), 7.03 (s, 1H, coumarin), 6.46 (s, 1H, coumarin), 5.18 (m, 2H, O—CH2), 4.64 (s, 2H), 3.3 (d, 2H), 3.0 (broad, 2H, CH₂).

A spiropyran L-glutamic acid (77) was used for synthesis of the corresponding N-carboxyanhydride of spiropyran L-glutamate (78) according to Scheme 14 as per the N-carboxyanhydride synthesis procedure presented by Poche et al., 1999. Spiropyran L-glutamate (77) (6.13 mmol) was suspended in dry ethyl acetate (50 mL) and the solution was heated to reflux. Triphosegene (2.04 mmol) was added and the reaction was refluxed for 4-5 hours under N₂. The reaction solution was cooled to room temperature and any unreacted spiropyran L-glutamate was removed by filtration. The reaction solution concentrated and was then cooled to 5° C. to form the final product (78) as yellowish crystals.

¹H NMR (CDCl3) δ=8.4 (s, 1H), 8.15 (s, 1H), 7.2 (m, 1H), 6.8 (m, 1H), 6.5 (m, 2H), 5.9 (d, 2H), 5.4 (broad, 1H, NH), 5.09 (m, 1H), 4.01 (m, 2H, CH₂), 3.59 (m, 2H, CH₂), 1.4 (s, 6H, CH₃).

### Example 4.1—Preparation of Polymeric Surface Using N-Carboxyanhydrides

As shown in Scheme 17, which provides a general scheme for synthesizing polymeric surfaces using N-carboxyanhydrides, the photo-switchable/responsive polypeptides were prepared by Ring Opening Polymerization initiated with aminopropyl triethoxysilane initiator in dry dimethylformamide (DMF). For synthesizing of amine terminated monolayer on silicon wafers, freshly diced (1×1 cm²) silicon wafers used as substrates were rinsed with Milli-Q water and ultrasonically cleaned with subsequently warm (30° C.) ethanol and dichloromethane (DCM.). After these precleaning steps, the silicon wafers treated with Pirahna solution (H₂O₂/H₂SO₄, 3:7) for 30 minute and followed by thoroughly washing with large quantity of Milli-Q water, again ultrasonically cleaned with methanol and methanol/toluene and toluene for 5 minutes. Then they were immediately silanized with freshly distilled (3-aminopropyl)triethoxy silane (2%) in dry toluene. The solution polymerizations were performed in anhydrous tetrahydrofuran (THF) at 55° C. in specially designed glassware for two days. The photo-responsive moiety modified amino acid NCA solution (0.5 mol/L) was added to the amine terminated silanized substrates. After the polymerizations, the films were washed with a mixture of dichloroacetic acid and chloroform (20/80 (v/v)) for 24 hours to remove any nongrafted material and subsequently with chloroform for 1 hour. The samples were dried in a vacuum before characterization.

### (a) Poly Naphthasene-Tyrosine (91)

Poly napthacene-tyrosine was prepared according to Scheme 19. For synthesizing of amine terminated monolayer on silicon wafers, freshly diced (1×1 cm²) silicon wafers used as substrates were rinsed with Milli-Q water and ultrasonically cleaned with subsequently warm (30° C.) ethanol and dichloromethane (DCM.). After these precleaning steps, the silicon wafers treated with Pirahna solution (H₂O₂/H₂SO₄, 3:7) for 30 minutes and followed by thoroughly washing with large quantity of Milli-Q water, again ultrasonically cleaned with methanol and methanol/toluene and toluene for 5 minutes. Then they were immediately silanized with freshly distilled (γ-aminopropyl)triethoxy silane (2%) in dry toluene. The solution polymerizations were performed in anhydrous tetrahydrofuran (THF) at 55° C. in specially designed glassware for two days. The photo-responsive naphthacene-Tyr-NCA (72) solution (0.5 mol/L) was added to the amine terminated silanized substrates. After the polymerizations, the films were washed with a mixture of dichloroacetic acid and chloroform (20/80) for 24 h to remove any non-grafted material and subsequently with chloroform for 1 hour.

### (b) Poly spiropyran L-glutamate (90)

Poly spiropyran L-glutamate was prepared according to Scheme 18. The surface-initiated polymerization of spiropyran L-glutamate N-carboxyanhydride (78) was performed in anhydrous tetrahydrofuran (THF) at 55° C. in specially designed glassware, initiated on (γ-aminopropyl)triethoxy silane grafted silicon wafer. The spiropyran L-glutamate N-carboxyanhydride (NCA) solution (0.5 mol/L) was added to the amine terminated silanized substrates in THF at 55° C. After the polymerizations, the films were washed with a mixture of dichloroacetic acid and chloroform (20/80) for 24 hours to remove any non-grafted material and subsequently with chloroform for 1 hour.

### (c) Poly (6-bromo-7-hydroxycoumarin-4-ylmethoxycarbonyl)-L-aspartate (93)

Poly aspartate bearing a coumarin-derived stimuli-switchable moiety was synthesized in accordance with Scheme 21. The surface-initiated polymerization of N-carboxyanhydride of 6-bromo-7-hydroxycoumarin-yl-methoxycarbonyl)-L-aspartate (81) was performed in anhydrous tetrahydrofuran (THF) at 55° C. in specially designed glassware, initiated on (γ-aminopropyl)triethoxy silane grafted silicon wafer. The N-carboxyanhydride of 6-bromo-7-hydroxycoumarin-yl-methoxycarbonyl)-L-aspartate (NCA) (81) solution (0.5 mol/L) was added to the amine terminated silanized substrates in THF at 55° C. After the polymerizations, the films were washed with a mixture of dichloroacetic acid and chloroform (20/80) for 24 h to remove any non-grafted material and subsequently with chloroform for 1 hour.

### (d) Poly 4,5-dimethoxy 2-nitrobenzyl cysteine (92)

A poly cysteine surface incorporating a nitrobenzyl stimuli-switchable moiety was prepared according to Scheme 20. The surface-initiated polymerization of N-carboxyanhydride of 4,5-dimethoxy 2-nitrobenzyl cysteine (75) was performed in anhydrous tetrahydrofuran (THF) at 55° C. in specially designed glassware, initiated on (γ-aminopropyl)triethoxy silane grafted silicon wafer. The N-carboxyanhydride of 4,5-dimethoxy 2-nitrobenzyl cysteine (NCA) solution (75) (0.5 mol/L) was added to the amine terminated silanized substrates in THF at 55° C. After the polymerizations, the films were washed with a mixture of dichloroacetic acid and chloroform (20/80) for 24 hours to remove any non-grafted material and subsequently with chloroform for 1 hour.

### (e) Formation of Silicon Surface Incorporating Polynitrobenzylglutamate

The modified nitrbenzylglutamate amino acid having the structure (83) was incorporated as a polymer on a silicon surface using the reaction scheme set out in Scheme 17.

### Example 5.0—AFM Characterization of Nanoparticle Switching

Atomic force microscopy is normally used to visualize the topographies of a polymer grafted onto the silicon surfaces. In order to find the hydrophobic-hydrophilic pattern by photo irradiation, a photo-switchable surface was prepared from napthacene methacrylate (24) using a pre-polymerization modification approach. The photo-switchable surface was placed on a cromium/glass mask and fixed with glass plate and clips. The substrate and mask were soaked in ultrapure water and irradiated with UV light having a wavelength of 350 nm. Finally, the patterned brush substrate was soaked ultrapure water and blown to dry. The surface morphologies of photo-responsive polymer brushes were performed in the “Tapping mode” obtained using Atomic force microscopy (using Bruker Icon AFM Santa Barbara, Calif.) system. Electrically conducting SCM-PIT probe (Bruker, Santa Barbara, Calif.) with resonance frequency 64.1 kHz, spring constant of 2.8 N/m, tip of radius of curvature (a) of 20 nm, and a quality factor of approximately 200 was used as the probe. The topography of the EFM images was processed using Nanoscope™ analysis software (V1.40, Bruker). To show the distribution of hydrophilic side of the patterned polymer surface, selective attachment of cationic silica nanoparticles is introduced. The particles are selected to be absorbed to the hydrophilic part (light irradiation area) of the patterned polymer surface by taking advantage of the counterion condensation behavior of the oppositely charged species.

Results are presented in FIG. 6. The upper left AFM image shows that there is no clear boundary between the photo-irradiated (hydrophilic) and non-irradiated (hydrophobic) portions of the surface (i.e. the right and left sides of the image) in the absence of the cationic silica nanoparticles. However after introducing cationic silica nanoparticles, which attach to the hydrophilic (photo-irradiated) portion only, a clear divide can be seen between the photo-irradiated (hydrophilic) side (right-hand side in the upper right image) and the non-irradiated (hydrophobic) side (left hand side of the upper right image). The lower image of FIG. 6 shows adhesion mapping of the upper right side image, and clearly demonstrates the hydrophilic-hydrophobic boundary. The results of this experiment demonstrate that a very clear hydrophobic-hydrophilic region can be defined by the use of light to switch a napthacene-based stimuli-switchable moiety.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

### REFERENCES

The following references are of interest to the subject matter described herein. Each of the following references is incorporated by reference herein in its entirety for all purposes.
Angiolini et al., “Optically active photochromic methacrylic polymers with controlled average molecular weight and defined end-groups by atom transfer radical polymerization”, Polymer, 2005, 46, 2424-2432.
Baumann et al., “Development of light-responsive porous polycarbonate membranes for controlled caffeine delivery”, RSC Adv., 2013, 3, 23317.
Chang et al., “Preparation and characterization of spironapthooxazine/polyacrylate photochromic hard coatings on plastic substrates”, J. Appl. Sci. Eng., 2014, 17(2), 167-174.
Ciardelli et al., “Photochromic polymers: effects on structure and environment on photoresponsiveness”, Pol. for Adv. Tech., 1995, 6, 32-41.
Klajn, “Spiropyran-based dynamic materials”, Chem. Soc. Rev., 2014, 43, 148.
Liu and Dong, “Photoresponsive poly(S-(o-nitrobenzyl)-L-cysteine)-b-PEO from a L-cysteine N-carboxyanhydride monomer: synthesis, self-assembly, and phototriggered drug release”, Biomacromolecules, 2012, 13, 1573-1583.
Lu et al., “Recent advances in amino acid N-carboxyanhydrides and synthetic polypeptides: chemistry, self-assembly and biological applications”, Chem. Commun., 2014, 50, 139.
D. S. Poche, M. J. Moore, J. L. Bowles, “An unconventional method for purifying the N-carboxyanhydride derivatives of γ-alkyl-L-glutamates” Synth. Commun. 1999, 29, 843.
Samanta and Locklin, “Formation of photochromic spiropyran polymer brushes via surface-initiated, ring-opening metathesis polymerization: reversible photocontrol of wetting behavior and solvent dependent morphology changes”, Langmuir, 2008, 24, 9558-9565.
Wang et al., “Fast photo-switched wettability and color of surfaces coated with polymer brushes containing spiropyran”, J. Appl. Polym. Sci., 2012, 125, 870-875.
Williamson, “Theory of aetherification”, Philosophical Magazine, 2009, 37, 350-356.
Yin et al., “Light-responsive helical polypeptides capable of reducing toxicity and unpacking DNA: toward nonviral gene delivery”, Angew. Chem. Int. Ed., 2013, 52, 9182-9186.
Zhang et al., “Photochromic spiropyran dendrimers: ‘click’ syntheses, characterization, and optical properties”, Macromol. Rapid Commns., 2008, 29, 193-201.
Zhu et al., “Spiropyran-based photochromic polymer nanoparticles with optically switchable luminescende”, J. Am. Chem. Soc., 2006, 128(13), 4303-4309.

1. A polymer comprising a plurality of monomeric units selected from the group consisting of pyranonaphtha chromenone derivatives, perylene chromenone deriviatives and lactone derivatives, wherein at least two of the monomeric units comprise a reversible stimuli-switchable moiety, wherein the polymer is reversibly switchable in response to an external stimuli, wherein the stimuli-switchable moiety undergoes structural isomerization upon exposure to the external stimuli, and wherein the external stimuli comprises light, heat, or chemical stimulus, and wherein the chemical stimulus optionally comprises a change in pH.

2. The polymer as defined in claim 1, wherein the polymer is switchable between a hydrophobic state and a hydrophilic state in response to the external stimuli, or is switchable between a hydrophilic state and a hydrophobic state in response to the external stimuli; and/or wherein the polymer is switchable between a colorless state and a colored state in response to the external stimuli, or is switchable between a colored state and a colorless state in response to the external stimuli.

3. The polymer as defined in claim 1, wherein the stimuli-switchable moiety comprises a pyranonaphtha chromenone derivative or a pyranoperylene chromenone having the structure (I) or (II):

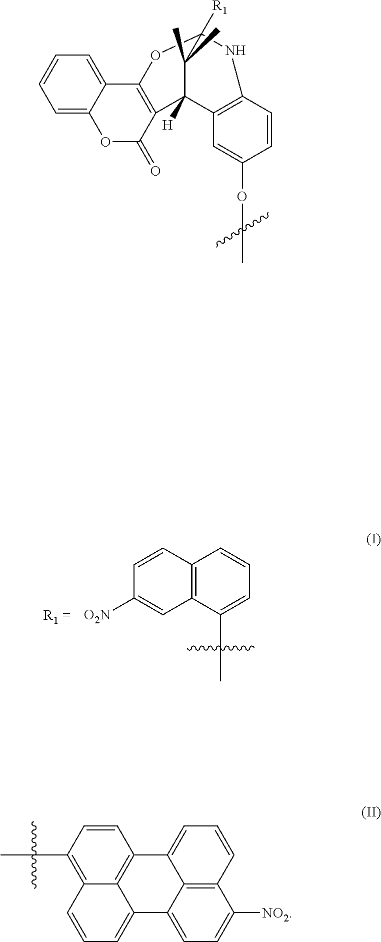

4. The polymer as defined in claim 3, wherein the stimuli-switchable moiety reversibly switches from hydrophobic to hydrophilic, upon exposure to light having a wavelength in the range of approximately 250 nm to 450 nm or approximately 650 nm to 800 nm.

5. The polymer as defined in claim 4, wherein the stimuli-switchable moiety reversibly switches from hydrophilic to hydrophobic, upon an increase in temperature to above approximately 50° C.

6. The polymer as defined in claim 4, wherein a degree of change in a contact angle of the polymer is at least 1.5 times greater than a degree of change in a contact angle of a corresponding polymer incorporating a nitrobenzene-derived stimuli-switchable moiety.

7. The polymer as defined in claim 1, wherein the monomeric units comprise lactone derivatives, and wherein the stimuli-switchable moiety comprises a spiropyran, a spirooxazine, an azobenzene, a bisthienylethene, a flugide, 2-diazo-1,2-napthoquinone-5-sulfonyl-methylacrylamide, or a pyrano aryl chromenone derivative, which is optionally a pyranobenzyl chromenone derivative, a pyranonaphtha chromenone derivative, or a pyranoperylene chromenone.

8. The polymer as defined in claim 1, wherein the monomeric units comprise lactone derivatives, and wherein:
the stimuli-switchable moiety comprises a pyrano aryl chromenone derivative, optionally a pyranobenzyl chromenone derivative, a pyranonaphtha chromenone derivative, or a pyranoperylene chromenone, and the stimuli comprises light having a wavelength in the range of about 250 nm to about 450 nm, or in the range of about 650 nm and 800 nm; or
the stimuli-switchable moiety comprises a spiropyran derivative, and the stimuli comprises light having a wavelength in the range of about 350 nm to about 550 nm.

9. (canceled)

10. (canceled)

11. (canceled)

12. The polymer as defined in claim 1, wherein the polymer is reversibly switchable between the hydrophilic and hydrophilic states.

13. The polymer as defined in claim 1, wherein the stimuli is selected from the group consisting of light and heat.

14. The polymer as defined in claim 1, wherein the stimuli-switchable moiety is a photochromic moiety.

15. The polymer as defined in claim 1, wherein the polymer is tethered to a solid surface.

16. The polymer as defined in claim 15, wherein the surface is selected from the group consisting of a microfluidic chip, a display device, a selective size separator, a transducer, a nanoparticle, and a membrane.

17. The polymer as defined in claim 15, wherein the surface is biocompatible.

18. (canceled)

19. (canceled)

20. (canceled)

21. (canceled)

22. (canceled)

23. (canceled)

24. A molecule comprising a stimuli-switchable moiety based on pyranonaphtha chromenone or pyranoperylene chromenone.

25. The molecule as defined in claim 24, wherein the stimuli-switchable moiety comprises the chemical structure (I) or (II): 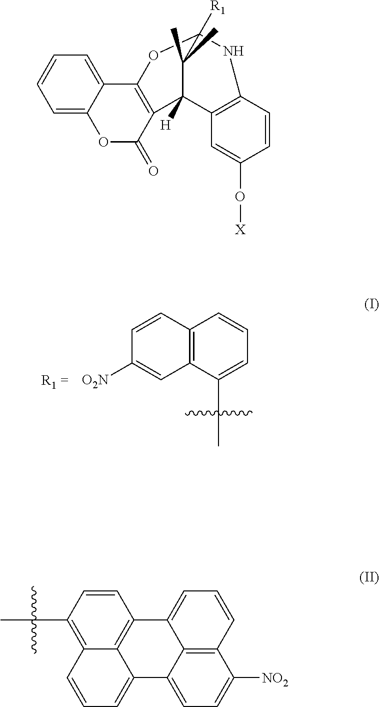
wherein X denotes a remaining portion of the molecule.

26. The molecule as defined in claim 25, wherein the stimuli-switchable moiety reversibly switches from hydrophobic to hydrophilic, upon exposure to light having a wavelength in the range of 250 nm to 450 nm or approximately 650 nm to 800 nm.

27. The molecule as defined in claim 25, wherein the stimuli-switchable moiety reversibly switches from hydrophilic to hydrophobic, upon an increase in temperature to above approximately 50° C.

28. The molecule as defined in claim 25, wherein the molecule comprises a monomer for synthesizing a polymer, wherein X comprises one of the following groups:

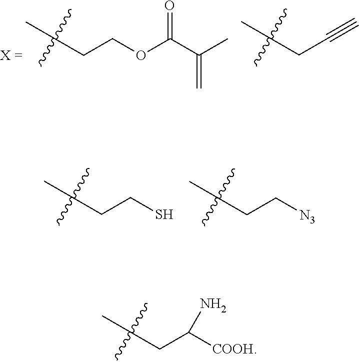

29. A polymerizable monomer having a reversible stimuli-switchable moiety, the monomer comprising a lactone, wherein the stimuli-switchable moiety is switchable in response to an external stimulus comprising light, heat, or chemical stimulus, wherein the chemical stimulus optionally comprises a change in pH, and wherein the stimuli-switchable moiety undergoes structural isomerization upon exposure to the external stimulus.

30.-45. (canceled)

46. A polymer as defined in claim 1, wherein the structural isomerization comprises cis-trans isomerization, trans-cis isomerization, ring-closing, or ring-opening.
